(19) United States
(12) Patent Application Publication
(10) Pub. No.: US 2022/0075098 A1
(43) Pub. Date: Mar. 10, 2022

(54) SYSTEMS AND METHODS OF TREATING A HYDROGEL-COATED MEDICAL DEVICE

(71) Applicant: TANGIBLE SCIENCE, INC., Redwood City, CA (US)

(72) Inventors: Kelly MABRY, Redwood City, CA (US); Karen L. HAVENSTRITE, Saratoga, CA (US); Brandon Mcnary FELKINS, San Francisco, CA (US); Victor Wayne MCCRAY, Redwood City, CA (US); Katharine GIFFORD, San Francisco, CA (US); Margaret WALTER, San Francisco, CA (US); Jessalyn IMDIEKE, Menlo Park, CA (US)

(73) Assignee: TANGIBLE SCIENCE, INC., Redwood City, CA (US)

(21) Appl. No.: 17/413,665

(22) PCT Filed: Dec. 17, 2019
(86) PCT No.: PCT/US2019/066851

§ 371 Date: Jun. 14, 2021

Related U.S. Application Data
(60) Provisional application No. 62/781,929, filed on Dec. 19, 2018.
(60) Provisional application No. 62/885,873, filed on Aug. 13, 2019.

(51) Int. Cl. G02B 1/18; G02B 1/04; C09D 133/26; C09D 171/08

(52) U.S. Cl. CPC G02B 1/18; C09D 171/08; C09D 133/26; G02B 1/043

(57) ABSTRACT

This present invention discloses kits, solutions, and methods for regenerating a hydrogel coating on a hydrogel coated medical device. The solution comprises a first hydrophilic polymer and a second hydrophilic polymer. The reaction of the first and second hydrophilic polymers with the originally coated surface of the medical device forms an at least partially covalently crosslinked hydrogel layer. The solution may be part of a kit, wherein the first and second hydrophilic polymers are provided separately with or without the medical device included in the kit. The originally coated surface of the medical device has an initial thickness before use or wear and a second thickness before use or wear, the second thickness being less than the first thickness. Treatment of the hydrogel coated medical device with the solutions described herein increased the second thickness to approach a final thickness that is substantially equal to the initial thickness.

### CROSS-REFERENCE TO RELATED APPLICATIONS

This application claims the priority benefit of U.S. Provisional Patent Application Ser. No. 62/781,929, filed Dec. 19, 2018; and U.S. Provisional Patent Application Ser. No. 62/885,873, filed Aug. 13, 2019, the contents of each of which are herein incorporated by reference in their entireties.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical devices and coatings, and more specifically to the field of treating or refreshing coated medical devices to form a thicker coating thereon. Described herein are systems and methods for such treatments.

### BACKGROUND

Contact lenses are often used for vision correction, as an alternative to glasses or vision correction surgery. They may also be used for ocular pathologies and non-medical or aesthetic purposes. Currently, contact lenses are typically classified as either “soft” or “hard” lenses. “Soft” lenses typically include hydrogel materials with greater than 30% water content. Soft lens hydrogels may be formed from traditional hydrophilic polymers or from silicone-containing polymers to enhance oxygen permeability. Soft lenses can also be composed of silicone elastomers, which have a low water content. “Hard” lenses are comprised of a rigid, gas-permeable material. “Hybrid” lenses combine a rigid center with a soft material skirting, circumferentially disposed around the rigid center.

Contact lenses may often be uncomfortable to wear as they become dry and an irritant in the eye, either over the course of daily wear, or over the lifetime of the lens. Silicone-based lenses have a hydrophobic core, which can lead to abrasion of ocular tissue and infection, rigid lenses are too hard to be comfortable and “hybrid” lenses, like soft and hard lenses, are prone to protein build-up.

Recent changes in contact lenses have attempted to address these issues. Substances and materials can be deposited onto a contact lens surface to improve the biocompatibility of the lens and therefore improve the interaction of the lens with the ocular region. For example, hydrophilic agents or wetting agents may be added to the lens solution to temporarily coat the lens such that upon wearing, the wetting angle of the lens is reduced. However, these temporary hydrophilic “coatings” are short-lived and quickly rinsed from the surface either with a cleaning solution or over the course of a day of wear.

Additionally, hydrophilic polymer solutions have been bound to the lens surface to create an outer coating on the lens surface that reduces the wetting angle and remains on the lens surface during rinsing with cleaning solution and through several or a plurality of wearing cycles. However, over time, these hydrophilic coatings are subject to wear, such that a thickness of the coating diminishes over the time and limits the useful life of the contact lens.

Further, with such treated lenses, there is a possibility for enhanced treatment of several pathologies, that include, but are not limited to, dry-eye disease, glaucoma, corneal ulcers, scleritis, keratitis, iritis, and corneal neovascularization. In particular, such lenses may be useful for preventing dry-eye like symptoms in contact lens wearers.

Dry eye disease is considered to be a consequence of a disruption of the tear film that covers the surface of the eye or a particular vulnerability to such disruption. This tear film is an aqueous layer disposed between an underlying mucous layer that is secreted by corneal cells, and an overlying lipid layer that is secreted by Meibomian glands on the conjunctival surface of the eyelids. The tear film includes an aqueous pool that transits across the eye surface, having a flow path that, to some degree, may be independent of the lipid layers that it is disposed between at any point in time. The improved lenses diminish or substantially eliminate contact lens disruption of the tear film.

Such lenses may lose their effectiveness over time as the coating is either rinsed from the surface or reduced over time (reduced thickness) as a result of continued wear or use. Some systems recommend placing drops in the eye to coat the lens with additional hydrophilic moieties; however, such systems are ineffective and incapable of truly regenerating the coating on the lens.

It is thus apparent that additional systems, devices, and methods may be contemplated to increase contact lens comfortability.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A shows a contact lens having a concave and convex surfaces.

FIG. 1B is a cross-sectional view of an exemplary contact lens with a covalently attached cross-linked hydrogel layer.

FIG. 2 is a cross-sectional view of the contact lens shown in FIG. 1B on the cornea.

FIG. 3 illustrates one embodiment of a system for treating or refreshing a medical device.

FIG. 4A shows schematically a hydrophilic polymer having two species covalently attached to a coated lens core.

FIG. 4B shows schematically another embodiment, of a hydrophilic polymer having two species covalently attached to a coated lens core.

FIG. 4C shows schematically another embodiment of a hydrophilic polymer having two species covalently attached to a coated lens core.

FIG. 5A shows schematically various reactants and reactions for regenerating a hydrophilic polymer coating on a previously coated medical device.

FIG. 5B shows schematically various exemplary branched and pendant reactive groups on a polymer.

FIGS. 6A-6C show various embodiments of a receptacle for treating or refreshing one or more medical devices therein.

FIG. 7 illustrates one embodiment of a kit for treating or refreshing one or more medical devices.

FIG. 8 illustrates various receptacles for containing a solution therein.

FIG. 9 illustrates a flow chart of one embodiment of a method of using a kit for treating or refreshing a medical device.

FIG. 10 shows schematically a contact lens with a hydrophilic polymer layer thereon having a first thickness (baseline) before wear or use, a second thickness after wear or use, and a third thickness that is substantially equal to the first thickness after treatment with a refreshing or rejuvenating solution.

FIG. 11 shows graphical data indicating that the rejuvenating or refreshing solution improves the advancing contact angle of a contact lens.

FIGS. 12A-12B show graphical data indicating that the rejuvenating or refreshing solution regenerates the hydrogel coating on the medical device substantially back to a baseline or initial thickness over time and after simulated wear, respectively.

FIG. 13 shows graphical data over time indicating that the rejuvenating or refreshing solution regenerates the hydrogel coating on the medical device substantially back to a baseline or initial thickness.

FIG. 14 shows a table of wetting and contact angle test results after refreshing or rejuvenating the coating on the contact lens.

FIG. 15 shows a table of biocompatibility test results for a regenerating or rejuvenating solution.

FIGS. 16A-16C show a captive bubble test.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

There is a need for a new and useful system and method for treating, retreating, or refreshing a specially coated medical device. In particular, there is a need for one or more systems, devices, and methods that regenerate the hydrophilic layer or polymer layer coating a medical device.

One aspect of the present disclosure is directed to a solution for rejuvenating, regenerating, or refreshing a hydrophilic polymer coating on a coated medical device, the solution includes a first hydrophilic polymer and a second hydrophilic polymer. In some embodiments, reaction of the first and second hydrophilic polymers with the surface of the medical device or an existing coating on the medical device forms an at least partially covalently crosslinked hydrogel layer to reduce the wetting angle of the medical device, e.g., a contact lens.

Another aspect of the present disclosure is directed to a solution for regenerating or refreshing or rejuvenating an existing coating on a medical device. In some embodiments, the solution includes a first hydrophilic polymer comprising one or more reactive functionalities; and a second hydrophilic polymer comprising one or more reactive functionalities. The first and second hydrophilic polymers react with an existing coating on a surface of the medical device as well as with each other to form a hydrophilic polymer layer that is at least partially covalently crosslinked between the first and second polymers in the solution and with the existing coating on the surface of the medical device.

As used herein, treating comprises retreating or refreshing or rejuvenating a previously coated medical device to regenerate the existing hydrogel or polymer layer or build upon an existing hydrogel or polymer layer or add to an existing hydrogel or polymer layer on the medical device. As used herein, treating, refreshing, rejuvenating, and regenerating may be used interchangeably.

As used herein, an existing coating or layer refers to a coating present on a surface of a medical device before rejuvenation or refreshing or retreating. The existing coating may have been deposited during the manufacturing phase, before the last use or wear cycle, after purchase, before a first use or wear, etc. The existing coating may comprise one or more polymers, for example, polyethylene glycol (PEG), phosphorylcholine, poly(vinyl alcohol), poly(vinylpyrrolidinone), poly(N-isopropylacrylamide) (PNIPAM), polyacrylamide (PAM), poly(2-oxazoline), polyethylenimine (PEI), poly(acrylic acid), acrylic polymers such as polymethacrylate, polyelectrolytes, hyaluronic acid, chitosan, chondroitin sulfate, alginate, hydroxypropylmethylcellulose, dextran, etc. The existing layer may be bonded to, attached to, or otherwise coupled to a surface of the medical device. For example, the existing coating or layer may be at least partially covalently linked, cross-linked, or covalently cross-linked to a surface of the medical device. The existing coating or layer may be hydrophilic or hydrophobic. The existing coating or layer may have an initial thickness before use or wear and a second or reduced thickness after use or wear. The reduced thickness may occur after one use or wear cycle or after n number of use or wear cycles (e.g., n equals 1 day, 1-3 days, 3-6 days, 1-5 days, 1-10 days, 7-14 days, 2-4 weeks, 1 month, etc.). After treatment with the solutions or kits to regenerate or rejuvenate the existing coating or layer, the second or reduced thickness may increase such that is approaches the initial thickness, exceeds the initial thickness, or substantially equals the initial thickness.

In some embodiments, the medical device is a contact lens adapted for placement in the eye. Exemplary, non-limiting lenses include contact lens, smart lenses, disposable soft lenses, daily lenses, extended wear lenses, spherical lenses, toric lenses, multifocal or bifocal lenses, diagnostic lenses, monovision lenses, conventional lenses, rigid gas permeable lenses, hard lenses, color variation lenses, opaque lenses, enhancers lenses, and visibility tinted lenses. For ease of the description herein, lenses will be commonly referred to herein as contact lenses.

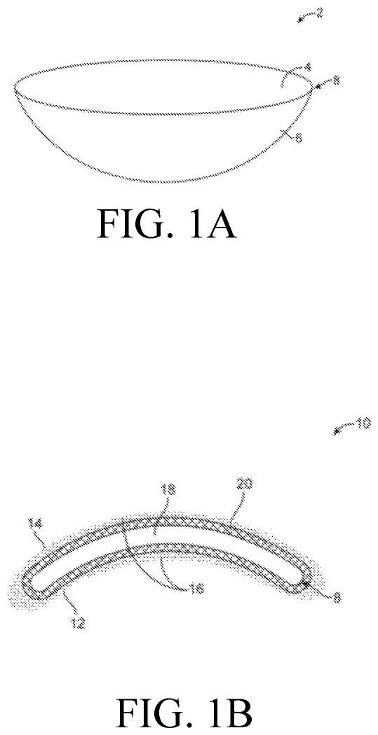

As shown in FIG. 1A, a contact lens 2 may be generally understood as having a body with a concave surface 4 and a convex surface 6. The lens body may include a periphery or a perimeter 8 between the surfaces. The periphery may also include a circumferential edge between the surfaces.

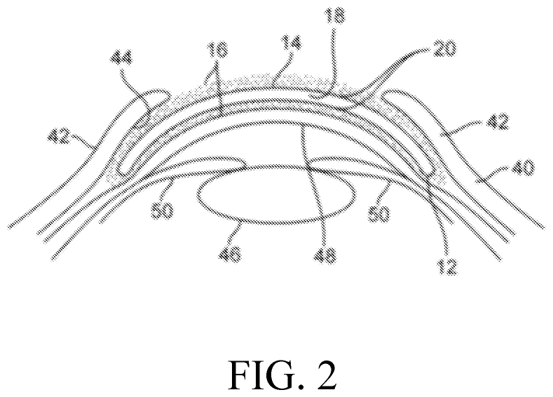

The concave surface 4 may also be referred to as a posterior surface and the convex surface 6 may also be referred to as an anterior surface, terms that refer to respective position when worn by a user. In practice, the concave surface of the lens is adapted to be worn against or adjacent to an ophthalmic surface. When worn, the concave surface may lie against a user's corneal surface 48 (see FIG. 2). The convex surface is outward-facing, exposed to the environment when the eye 40 is open. When the eye 40 is closed, the convex surface is positioned adjacent or against the inner conjunctival surface 44 of the eyelids 42 (see FIG. 2).

Because the convex and concave surfaces of a lens may be placed against or adjacent ophthalmic tissue such as the corneal surface, the properties of the surfaces can greatly affect a user's comfort and wearability of the lens as described above. For example, the lens may disrupt the tear film 16 of the eye 40 causing symptoms associated with dry eye. As such, embodiments described herein provide for a rejuvenated coated contact lens having a hydrophilic polymer rejuvenated on at least one of the lens's surfaces to improve the lens's wettability and wearability with minimal tear film disruption.

In one embodiment, the contemplated rejuvenated coated contact lens includes a core or bulk material with at least one surface having a hydrophilic polymer layer. In some cases, the rejuvenated hydrophilic layer is adapted for placement against an ophthalmic surface. The rejuvenated hydrophilic layer may cover a portion of the lens core surface. Alternatively, the rejuvenated hydrophilic layer may completely or substantially completely cover the core surface.

In other variations, more than one core surface has a rejuvenated hydrophilic layer. For example, a hydrophilic polymer layer on both the concave and the convex surfaces of the lens may be rejuvenated. Each rejuvenated hydrophilic layer on either concave or convex surfaces may independently completely or partially cover respective surfaces. In some cases, the rejuvenated layer on each side of the core forms a contiguous hydrophilic layer across both surfaces.

In additional variations, the rejuvenated hydrophilic polymer layer is formed from a cross-linked hydrogel polymer network having one or more cross-linked species. The rejuvenated hydrophilic polymer network may be partially cross-linked or substantially fully cross-linked between the polymers in the rejuvenated layer and with one or more polymers or reactive groups in an existing coating on the lens. In some variations, the rejuvenated hydrophilic polymer is cross-linked to approximately 95% end group conversion. In some embodiments, the one or more polymers or reactive groups are chemically cross-linked. In some embodiments, the one or more polymers or reactive groups are ionically cross-linked. In some embodiments, the one or more polymers or reactive groups are physically cross-linked. In some embodiments, the one or more polymers or reactive groups can be cross-linked via a combination of chemical cross-linking, physical cross-linking, and ionic cross-linking.

Referring to FIG. 1B, a cross-section of an exemplary embodiment of a rejuvenated coated contact lens 10 is shown. Rejuvenated coated contact lens 10 includes a lens core 18 and a rejuvenated hydrophilic polymer layer 20 attached to the core 18. As shown, a rejuvenated hydrophilic polymer layer 20 surrounds the core 18. Both the concave and convex surfaces 12, 14 are coated by the same rejuvenated hydrophilic polymer layer 20 on both sides of the lens 18 with the hydrophilic polymer layer 20 extending to the peripheral edge 8 of the core 10. As shown, the outer hydrophilic layer 20 is substantially contiguous through or across a circumferential edge portion 18. A variety of different materials can be used as the lens core as described in detail below. In some embodiments, the lens core can be a rigid gas permeable material. In some embodiments, the lens core can be a hydrophobic material, such as silicone. As used herein, silicone includes polysiloxanes. In some embodiments, the lens core can include a hydrogel.

Returning to FIG. 2, the rejuvenated coated contact lens 10 of FIG. 1B is positioned in a user's eye 40. The eye 40 is shown with eye lens 46 and iris 50. The concave surface 12 of the lens 10 is disposed and centered on the cornea. The convex surface 14 of the lens 10 is directed outwardly, facing the environment when the eye 40 is open. When the eyelid 42 closes, the convex surface 14 is adjacent to the inner or conjunctival surface 44 of the eyelid 42. As the eyelids 42 open and close, the conjunctival surface 44 slides across the convex surface 14 of the lens 10.

When placed on the cornea, the rejuvenated hydrophilic layer 20 of the contact lens 10 interacts with the natural tear film 16 of the eye 40. The contact lens 10 may be positioned within the tear film 16 and/or substantially reside within the aqueous layer of the tear film 16 that covers the eye 40. In some cases, the lens 10 is immersed in the tear film 16. The rejuvenated hydrophilic layer may be adapted to minimize disruption of the tear film by the contact lens.

In some embodiments, the medical device is one or more coated contact lenses. The disclosed solutions, kits, and methods may be used to treat or retreat coated hard, soft, and/or hybrid contact lenses, although any suitable medical device or contact lens may be envisioned.

The contact lens envisioned may be any suitable contact lens, and in the present aspect, the surface of the contact lens comprises a hydrophilic polymer layer. A coated contact lens includes a lens core and a hydrophilic polymer layer attached to the core. A hydrophilic polymer layer surrounds the core. Both the concave and convex surfaces are coated by the same hydrophilic polymer layer on both sides of the lens with the hydrophilic polymer layer extending to the peripheral edge of the core. As shown, the outer hydrophilic layer is substantially contiguous through or across a circumferential edge portion. A variety of different materials can be used as the lens core. In some embodiments, the lens core is a rigid gas permeable material. In some embodiments, the lens core is a hydrophobic material, such as silicone, which may include polysiloxanes. In some embodiments, the lens core comprises a soft lens material. In some embodiments, the lens core can include a hydrogel.

Any suitable contact lens with any lens core may be used, for example any lens core having an existing coating or layer. For example, the lens core itself may be hydrophobic or hydrophilic. A hydrophilic core may have adequate water content but lack protein binding resistance that is imparted by the contemplated hydrophilic layer. A hydrophilic core may include a hydrogel containing core such as a pure hydrogel lens. For example, the core may contain Polyhexyethyl methacrylate lenses (pHEMA).

In some embodiments, the lens core is a rigid gas permeable (RGP) material. In some embodiments the rigid gas permeable material is non-hydrophilic. In some embodiments the rigid gas permeable material is hydrophobic. Examples of rigid gas permeable materials include: cellulose acetate butyrate, polyacrylate-silicone, non-hydrophilic silicone elastomers, polysiloxane, fluoro-silicon polymers, etc. As used herein silicone includes polysiloxanes. Examples of commercial RGP lenses that can be treated with the processes disclosed herein include: Bausch & Lomb Boston Lens, Paragon CRT lens, Menicon Rose K, Menicon Lagado Flosi, Menicon Lagado Tyro, Menicon Lagado Onsi, Contamac Optimum Classic, Contamac Optimum Comfort, Contamac Optimum Extra, Contamac Optimum Extreme, Contamac Optimum Infinite, HEXA100, ENFLU 18, Fluorperm 92, Fluoroperm 60/Paragon HDS, Fluoroperm 30/Paragon Thin, Fluoroperm 151/HDS 100, Boston XO, Boston XO2, Boston ES, and Boston EO. The hydrophilic coatings described herein can be formed on one or both of the convex and concave surfaces of the RGP core as described herein.

The rigid lens can be described based on the lens modulus. For example, rigid lenses typically have a modulus of about 2000 MPa (2 GPa). In contrast, a soft lens has a modulus on the order of about 2 MPa or less. In some embodiments, a rigid lens core can be described as having an elastic modulus of greater than 500 MPa.

In some embodiments, the rigid gas permeable material comprises fluorine. In some embodiments, the rigid gas permeable material comprises a fluoro-acrylate. In some embodiments, the lens core can be made of materials other than silicone and can be substantially free of silicone and polysiloxanes.

The water equilibrium content can be described as the amount of water absorbed by the lens or lens core at equilibrium. For example, the water equilibrium content can be determined by weighing the dehydrated lens core or lens, submerging the lens in water for several minutes, removing the lens from the water, and weighing the lens after being submerged in the water. The water equilibrium content can then be calculated by subtracting the dry weight of the lens from the weight of the lens after the water bath and dividing that value by the dry weight. The water equilibrium content can be expressed as a percentage.

In some embodiments, the rigid gas permeable lens core has a water equilibrium content of less than about 5%. In some embodiments, the rigid gas permeable lens core has a water equilibrium content of less than about 4%. In some embodiments, the rigid gas permeable lens core has a water equilibrium content of less than about 3%. In some embodiments, the rigid gas permeable lens core has a water equilibrium content of less than about 2%. In some embodiments, the rigid gas permeable lens core has a water equilibrium content of less than about 1%. In some embodiments, the rigid gas permeable lens core has a water equilibrium content of less than about 0.5%. In some embodiments, the rigid gas permeable lens core has a water equilibrium content of less than about 0.1%.

In some embodiments, the lens core includes a rigid gas permeable material in a center region with a soft outer skirt circumferentially disposed about the center region, the soft outer skirt comprising silicone or other soft material. RGP lenses having a soft outer skirt are known as hybrid lenses. The original coatings and regenerated or rejuvenated coatings described herein can be formed on one or both of the convex and concave surfaces of the hybrid RGP/soft coating core, as described herein. Examples of commercial hybrid RGP lenses include those made by Synergeyes, such as the Synergeyes Duette Lens and the Synergeyes Ultra Health, and Laboratoire LCS, such as EyeBrid.

The RGP and hybrid RGP lenses are typically used by the patient for several months or more. In some cases, the RGP and hybrid RGP lenses can be used for a year or more. In contrast to the soft lenses, which are disposable and used for shorter amounts of time, the RGP and hybrid RGP lenses can be exposed to harsher cleaning processes than the disposable soft lenses. In order to meet the design requirements for RGP lenses and hybrid RGP lenses, it is desirable for any coatings to have a sufficiently long shelf life as well as the capability to withstand the more rigorous cleaning associated with those types of lenses. Alternatively, the coating may be regenerated multiple times throughout the wearing cycle by adding a rejuvenating solution comprising one or more reactive polymers to the lens, as described elsewhere herein.

Historically, hydrophilic layers, such as PEG were not considered to have good long-term stability. In co-owned application Ser. No. 13/975,868 filed on Aug. 26, 2013, PEG layers formed on soft core lenses were analyzed with accelerated aging studies. The aging studies indicated that the PEG layers had better than expected shelf life and stability, likely, at least, due to the thinness (e.g., less than 100 nm, less than 50 nm, 25 nm to 100 nm, etc.) of the PEG layers. The longevity of the coating with longer wear and more rigorous cleaning was unexpected. Additional testing has shown that the coating processes work well with RGP and hybrid RGP lenses. In addition, the coatings have demonstrated a suitable shelf life for RGP and hybrid RGP lenses even with exposure to the more rigorous cleaning processes associated with those lenses. The rate of wear is strongly patient-dependent (e.g., some patients clean their lenses more aggressively, wear their lenses longer, or have ocular surface conditions that lead to increased wear). The regenerating or rejuvenating coating processes, solutions, and kits described herein are substantially independent of such differences in wear. For example, as described elsewhere herein, regardless of the degree or uniformity of wear, the regenerating or rejuvenating solutions described herein are configured to regenerate the original coating substantially back to a baseline thickness or at least approach a baseline thickness or even exceed a baseline thickness. Other parameters, such as contact angle, elastic modulus, water content, etc. may also be regenerated substantially back to a baseline level or at least approach or even exceed a baseline level.

Additional details for the testing of the coatings through autoclave sterilization and accelerated aging tests are detailed in the examples.

In some embodiments, a lens core comprising silicone can be used with any of the hydrogel coatings described herein. The silicone lens core can comprise one or more polysiloxane compounds. In some embodiments, the polysiloxanes are cross-linked.

In some embodiments, the lens core can be primarily made of cross-linked polysiloxanes with trace impurities or trace additives. The lens core may consist substantially entirely of pure silicone (e.g. polysiloxane compounds), i.e. the core comprises about 100% silicone by weight. In other embodiments, the lens core can be made out of only polysiloxanes (e.g. 100% silicone by weight). In some embodiments, the lens core consists of polysiloxane. In other cases, the lens core, base, or substrate comprises about 10% to about 50% of silicone by weight. In some cases, the substrate or core comprises about 25% silicone by weight.

The silicone lens cores are resistant to water and do not absorb water. The lack of absorption of water can be described as the water equilibrium constant. In contrast to hydrogels, which by definition absorb water, silicone does not appreciably absorb water.

In some embodiments, a silicone lens core has a water equilibrium content of less than about 5%. In some embodiments, a silicone lens core has a water equilibrium content of less than about 4%. In some embodiments, a silicone lens core has a water equilibrium content of less than about 3%. In some embodiments, a silicone lens core has a water equilibrium content of less than about 2%. In some embodiments, a silicone lens core has a water equilibrium content of less than about 1%. In some embodiments, a silicone lens core has a water equilibrium content of less than about 0.5%. In some embodiments, a silicone lens core has a water equilibrium content of less than about 0.1%. In some embodiments, a lens core is substantially free of water.

In some embodiments, the lens core is a soft contact lens. For example, a soft contact lens can include an elastic modulus of less than about 2.0 MPa. In some embodiments, the lens core has an elastic modulus of less than about 1.8 MPa. In some embodiments, an elastic modulus of a hydrogel coated contact lens before use and/or wear is substantially equal to an elastic modulus of a regenerated or rejuvenated hydrogel coating on a hydrogel coated contact lens.

Conventional silicone contact lenses are known in the art to stick to the surface of the eye and are unsuitable for use in adults without additional processing and treatments. An uncoated conventional silicone lens can stick to the eye and damage the surface of the eye if the lens is moved or removed. The hydrogel coatings described herein can be used to coat both sides of silicone lenses to improve the lens properties and biocompatibility with the eye. The hydrogel coatings described herein allow the coated silicone lens to be adapted for on-eye movement without damage to the eye or ophthalmic surface. The coated lenses described herein are adapted to provide adequate on eye movement while maintaining the health of the ophthalmic surface and wearer comfort.

Another advantage of a silicone lens core is the high refractive index of silicone. Conventional hydrogel lenses have a much higher water content. The water content decreases the overall refractive index of the lens. A thin silicone core with a high refractive index can be used with a thin hydrogel coating to produce a contact lens with smaller thickness than conventional lenses and a higher refractive index. In some embodiments, the contact lens can have a refractive index of greater than about 1.420. The contact lens can have a thickness of less than 50 microns. In some embodiments, the contact lens has a thickness of less than 25 microns.

The on-eye movement can also be expressed as ionoflux as described in U.S. Pat. Nos. 5,760,100 and 5,849,811, which also describe methods for determining the ionoflux. With a silicone core, the ionoflux is very small since the silicone core would stick to the surface of the eye. In some embodiments, the lens core has an ionoflux diffusion coefficient of zero. In some embodiments, the lens core has an ionoflux diffusion coefficient of less than about 1×10⁻⁷ cm²/min.

The silicone cores can be formed from and include a variety of different monomers. Examples of preferred silicone-containing vinylic monomers include, without limitation, N-[tris(trimethylsiloxy)silylpropyl]-(meth)acrylamide, N-[tris(dimethylpropylsiloxy)-silylpropyl]-(meth)acrylamide, N-[tris(dimethylphenylsiloxy)silylpropyl](meth)acrylamide, N-[tris(dimethylethylsiloxy)silylpropyl](meth)acrylamide, N-(2-hydroxy-3-(3-(bis(trimethylsilyloxy)methylsilyl)propyloxy)propyl)-2-methyl acrylamide; N-(2-hydroxy-3-(3-(bis(trimethylsilyloxy)methylsilyl)propyloxy)propyl)acrylamide; N,N-bis[2-hydroxy-3-(3-(bis(trimethylsilyloxy)methylsilyl)propyloxy)propyl]-2-methyl acrylamide; N,N-bis[2-hydroxy-3-(3-(bis(trimethylsilyloxy)methylsilyl)propyloxy)propyl]acrylamide; N-(2-hydroxy-3-(3-(tris(trimethylsilyloxy)silyl)propyloxy)propyl)-2-methyl acrylamide; N-(2-hydroxy-3-(3-(tris(trimethylsilyloxy)silyl)propyloxy)propyl)acrylamide; N,N-bis[2-hydroxy-3-(3-(tris(trimethylsilyloxy)silyl)propyloxy)propyl]-2-methyl acrylamide; N,N-bis[2-hydroxy-3-(3-(tris(trimethylsilyloxy)silyl)propyloxy)propyl]acrylamide; N-[2-hydroxy-3-(3-(t-butyldimethylsilyl)propyloxy)propyl]-2-methyl acrylamide; N-[2-hydroxy-3-(3-(t-butyldimethylsilyl)propyloxy)propyl]acrylamide; N,N-bis[2-hydroxy-3-(3-(t-butyldimethylsilyl)propyloxy)propyl]-2-methyl acrylamide; N,N-bis[2-hydroxy-3-(3-(t-butyldimethylsilyl)propyloxy)propyl]acrylamide; 3-methacryloxy propylpentamethyldisiloxane, tris(trimethylsilyloxy)silylpropyl methacrylate (TRIS), (3-methacryloxy-2-hydroxypropyloxy)propylbis(trimethylsiloxy)methylsilane), (3-methacryloxy-2-hydroxypropyloxy)propyltris(trimethylsiloxy)silane, 3-methacryloxy-2-(2-hydroxyethoxy)-propyloxy)propylbis(trimethylsiloxy)methylsilane, N-2-methacryloxyethyl-O-(methyl-bis-trimethylsiloxy-3-propyl)silylcarbamate, 3-(trimethylsilyl)propylvinyl carbonate, 3-(vinyloxycarbonylthio)propyl-tris(trimethyl-siloxy)silane, 3-[tris(trimethylsiloxy)silyl]propylvinyl carbamate, 3-[tris(trimethylsiloxy)silyl]propyl allyl carbamate, 3-[tris(trimethylsiloxy)silyl]propyl vinyl carbonate, t-butyldimethyl-siloxyethyl vinyl carbonate; trimethylsilylethyl vinyl carbonate, and trimethylsilylmethyl vinyl carbonate). Most preferred siloxane-containing (meth)acrylamide monomers of formula (1) are N-[tris(trimethylsiloxy)silylpropyl]acrylamide, TRIS, N-[2-hydroxy-3-(3-(t-butyldimethylsilyl)propyloxy)propyl]acrylamide, or combinations thereof.

A class of preferred silicone-containing vinylic monomers or macromers is polysiloxane-containing vinylic monomers or macromers. Examples of such polysiloxane-containing vinylic monomers or macromers are monomethacrylated or monoacrylated polydimethylsiloxanes of various molecular weight (e.g., mono-3-methacryloxypropyl terminated, mono-butyl terminated polydimethylsiloxane or mono-(3-methacryloxy-2-hydroxypropyloxy)propyl terminated, mono-butyl terminated polydimethylsiloxane); dimethacrylated or diacrylated polydimethylsiloxanes of various molecular weight; vinyl carbonate-terminated polydimethylsiloxanes; vinyl carbamate-terminated polydimethylsiloxane; vinyl terminated polydimethylsiloxanes of various molecular weight; methacrylamide-terminated polydimethylsiloxanes; acrylamide-terminated polydimethylsiloxanes; acrylate-terminated polydimethylsiloxanes; methacrylate-terminated polydimethylsiloxanes; bis-3-methacryloxy-2-hydroxypropyloxypropyl polydimethylsiloxane; N,N,N′,N′-tetrakis(3-methacryloxy-2-hydroxypropyl)-alpha,omega-bis-3-aminopropyl-polydimethylsiloxane; polysiloxanylalkyl (meth)acrylic monomers; siloxane-containing macromer selected from the group consisting of Macromer A, Macromer B, Macromer C, and Macromer D described in U.S. Pat. No. 5,760,100 (herein incorporated by reference in its entirety); the reaction products of glycidyl methacrylate with amino-functional polydimethylsiloxanes; hydroxyl-functionalized siloxane-containing vinylic monomers or macromers; polysiloxane-containing macromers disclosed in U.S. Pat. Nos. 4,136,250, 4,153,641, 4,182,822, 4,189,546, 4,343,927, 4,254,248, 4,355,147, 4,276,402, 4,327,203, 4,341,889, 4,486,577, 4,543,398, 4,605,712, 4,661,575, 4,684,538, 4,703,097, 4,833,218, 4,837,289, 4,954,586, 4,954,587, 5,010,141, 5,034,461, 5,070,170, 5,079,319, 5,039,761, 5,346,946, 5,358,995, 5,387,632, 5,416,132, 5,451,617, 5,486,579, 5,962,548, 5,981,675, 6,039,913, and 6,762,264 (here incorporated by reference in their entireties); polysiloxane-containing macromers disclosed in U.S. Pat. Nos. 4,259,467, 4,260,725, and 4,261,875 (herein incorporated by reference in their entireties). Di and triblock macromers consisting of polydimethylsiloxane and polyalkyleneoxides could also be of utility. For example, one might use methacrylate end capped polyethyleneoxide-block-polydimethylsiloxane-block-polyethyleneoxide to enhance oxygen permeability. Suitable monofunctional hydroxyl-functionalized siloxane-containing vinylic monomers/macromers and suitable multifunctional hydroxyl-functionalized siloxane-containing vinylic monomers/macromers are commercially available from Gelest, Inc, Morrisville, Pa.

In some embodiments, the lens core may comprise a silicone-hydrogel (SiHy). The silicone hydrogel lens core can have a higher water content than the silicone lens core embodiments since hydrogels absorb water. For example, the silicone hydrogel lens core can have an equilibrium water content greater than 2% and less than 60%. In such cases, the SiHy lens core can be coated by the described hydrophilic polymer layers to improve wettability and wearability of the lens core. In other variations, the core comprises about 10% to about 50% of silicone by weight. In some embodiments, the hydrophilic layer can have a thickness of less than 100 nm.

In an exemplary embodiment, the silicone-containing layer or core of the coated contact lens is lotrafilcon, balafilcon, galyfilcon, senofilcon, narafilcon, omafilcon, comfilcon, enfilcon, or asmofilcon. In some cases, the silicone-containing core is NuSil Med 6755.

Alternatively, a non-silicone based core may be used as the substrate for coating. For example, an oxygen permeable lens made from a non-silicone material may also be coated with the described hydrophilic layer.

In an exemplary embodiment, the thickness of the core or core layer is from about 25 microns to about 200 microns, or from about 50 microns to about 150 microns, or from about 75 microns to about 100 microns, or from about 20 microns to about 80 microns, or from about 25 microns to about 75 microns, or from about 40 microns to about 60 microns.

In some embodiments, the medical device is not a contact lens. In such embodiments, the medical device may be configured to be implantable within a mammalian body. In a non-limiting example, the medical device is a stent configured to keep a cavity open. In another non-limiting example, the stent is configured to keep a blood vessel, bile duct, intestine, nasal passage or cavity, sinus cavity, or intraocular channel open.

In some embodiments, the medical device is a sensor, camera, vital sign monitor, drug depot device, neurostimulator, ultrasound, silicone implant, saline implant, hernia mesh, penile implant, intrauterine device, orthopedic rod or plate or pin or nails, pacemaker, cardiac valve, ear tube, aneurysm coil, or intraocular lens.

In some embodiments, the medical device is a test strip. Various non-limiting examples includes a drug, salivary, urine, blood, interstitial fluid, genetic testing, or semen test strip.

In some embodiments, the medical device is a tool configured to be inserted within a mammalian body. Various non-limiting examples include a catheter, trocar, endoscope, probe (e.g., interstitial, microdialysis, etc.), or laparoscope.

In some embodiments, the medical device is configured to be used externally on a mammalian body, for example, for use as a bandage, wound dressing, external sensor, hearing aid, or artificial skin.

In some embodiments, an outer surface of the medical device comprises one or more of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, silicone, polysiloxane, or silk.

In some embodiments, an outer surface of the device consists essentially of a material selected from the group consisting of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, silicone, polysiloxane, or silk.

In any of the preceding embodiments, a type of material or a composite of materials comprising the medical device determines a type of reactive group or combination of reactive groups required to covalently link or otherwise couple a hydrogel layer to an existing surface or coated surface of the medical device.

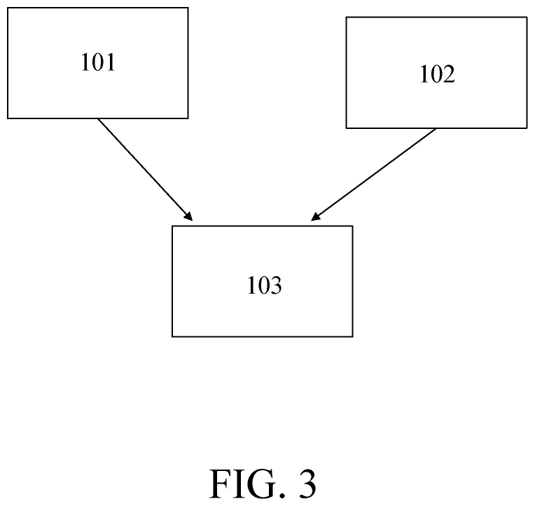

As shown in FIG. 3, a solution for treating or refreshing or rejuvenating a coated medical device comprises a first polymer 101 and a second polymer 102. In some embodiments, the first polymer comprises a reactive electrophilic group or a reactive nucleophilic group and the second polymer comprises a reactive electrophilic group or a reactive nucleophilic group complementary to the first polymer. The reactive electrophilic group and the reactive nucleophilic group are adapted to react to thereby form cross-links between the first polymer and the second polymer. In some embodiments, the first polymer comprises one or more reactive functionalities and the second polymer comprises one or more reactive functionalities.

Any suitable polymers may be used for the hydrophilic polymer population in the hydrophilic layer. In some cases, the polymer population includes species derived from polyethylene glycol (PEG), phosphorylcholine, poly(vinyl alcohol), poly(vinylpyrrolidinone), poly(N-isopropylacrylamide) (PNIPAM), polyacrylamide (PAM), poly(2-oxazoline), polyethylenimine (PEI), poly(acrylic acid), acrylic polymers such as polymethacrylate, polyelectrolytes, hyaluronic acid, chitosan, chondroitin sulfate, alginate, hydroxypropylmethylcellulose, and dextran.

In some embodiments, the hydrophilic polymer population includes a first species comprising polyethylene glycol (PEG). The first species comprising PEG can be combined with a second polymer species selected from the following non-limiting list: polyethylene glycol (PEG), phosphorylcholine, poly(vinyl alcohol), poly(vinylpyrrolidinone), poly(N-isopropylacrylamide) (PNIPAM), polyacrylamide (PAM), poly(2-oxazoline), polyethylenimine (PEI), poly(acrylic acid), acrylic polymers such as polymethacrylate, polyelectrolytes, hyaluronic acid, chitosan, chondroitin sulfate, alginate, hydroxypropylmethylcellulose, and dextran. In some embodiments, the second species can comprise a second PEG species. In some embodiments, the second species can comprise polyacrylamide.

In some embodiments, the hydrophilic polymer population includes a species comprising polyacrylamide. The species comprising polyacrylamide can be combined with a second polymer species selected from the following, non-limiting list: polyethylene glycol (PEG), phosphorylcholine, poly(vinyl alcohol), poly(vinylpyrrolidinone), poly(N-isopropylacrylamide) (PNIPAM), polyacrylamide (PAM), poly(2-oxazoline), polyethylenimine (PEI), poly(acrylic acid), acrylic polymers such as polymethacrylate, polyelectrolytes, hyaluronic acid, chitosan, chondroitin sulfate, alginate, hydroxypropylmethylcellulose, and dextran. In some embodiments, the second species can comprise a PEG species. In some embodiments, the second species can comprise a polyacrylamide species.

For example, in some embodiments, the first polymer is polyethylene glycol and the second polymer is polyacrylamide. Further for example, in some embodiments, the first and second polymers are polyacrylamide. In another example, the first and second polymers are polyethylene glycol.

In some embodiments, a hydrophilic polymer population includes one species, for example PEG. For example, each PEG species may comprise one or more or a plurality of pendant or branched reactive groups, for example, amine, carboxyl, hydroxyl, sulfonyl, mercapto, thiol, ketone, aldehyde, amide, ester, sulfite, phosphite, and any other reactive group. In some embodiments, each PEG species or population of PEG species includes a first reactive group in excess of a second reactive group. For example, a ratio of the first reactive group to the second reactive group may be 1:1; 1:5, 1:10, 1:15, 1:20, 1:25, 1:30, 1:35, 1:40, 1:45, 1:50, 1:55, 1:50, 1:55, 1:60, 1:65, 1:70, 1:75, 1:80, 1:85, 1:90, 1:95, 1:100, 1:150, 1:200, 1:250, 1:300, etc. In some embodiments, a ratio of the first reactive group to the second reactive group is between 1:1 and:100, 1:1 and 1:50, 1:20 and 1:40, etc.

In some embodiments, a hydrophilic polymer population includes one species, for example polyacrylamide. For example, each polyacrylamide species may comprise one or more or a plurality of pendant or branched reactive groups, for example, amine, carboxyl, hydroxyl, sulfonyl, mercapto, thiol, ketone, aldehyde, amide, ester, sulfite, phosphite, and any other reactive group. In some embodiments, each polyacrylamide species or population of polyacrylamide species includes a first reactive group in excess of a second reactive group. For example, a ratio of the first reactive group to the second reactive group may be 1:1; 1:5, 1:10, 1:15, 1:20, 1:25, 1:30, 1:35, 1:40, 1:45, 1:50, 1:55, 1:50, 1:55, 1:60, 1:65, 1:70, 1:75, 1:80, 1:85, 1:90, 1:95, 1:100, 1:150, 1:200, 1:250, 1:300, etc. In some embodiments, a ratio of the first reactive group to the second reactive group is between 1:1 and:100, 1:1 and 1:50, 1:20 and 1:40, etc.

The first polymer solution 101 (comprising the first polymer) and the second polymer solution 102 (comprising the second polymer) are physically combined to form solution 103. In some embodiments, there is no buffering agent present in the solution. In some embodiments, the solution has a pH of 7-11, 8-9, 7-9, 8-8.5, 8.2-8.6, 9.5-10.5, 9-11, or any range or subrange therebetween. In one embodiment, the pH is substantially 8, 8.5, 9, 9.5, 10, or 10.5. In some embodiments, the pH is less than 11. Alternatively, in some embodiments, the solution may further comprise a buffering agent such as triethanolamine and/or a solvent such as water.

In some embodiments, the one or more reactive functionalities include, but are not limited to: sulfonyl groups, amino-reactive groups, sulfhydryl-reactive groups, carboxyl groups, hydroxyl groups, haloalkyl groups, dienophile groups, aldehyde or ketone groups, alkenes, epoxides, phosphoramidites, amine groups, sulfhydryl groups, and a combination thereof.

In some embodiments, the reactive electrophilic groups are selected from the group consisting of amino-reactive groups, sulfhydryl-reactive groups, carboxyl groups, hydroxyl groups, haloalkyl groups, dienophile groups, aldehyde or ketone groups, alkenes, epoxides, phosphoramidites, and a combination thereof.

In some embodiments, the reactive nucleophilic group are selected from the group consisting of amines, amino-reactive groups, sulfhydryl, sulfhydryl-reactive groups, carboxyl groups, hydroxyl groups, haloalkyl groups, dienophile groups, aldehyde or ketone groups, alkenes, epoxides, phosphoramidites, and a combination thereof.

For example, a first reactive electrophilic group of the first polymer may react with a reactive nucleophilic group of the second polymer to at least partially covalently link the first polymer to the second polymer, and a second reactive electrophilic group, the same or different than the first reactive electrophilic group, may react with the existing layer on the lens core to covalently bond or link the first polymer to the existing layer on the lens core.

Further for example, the first reactive electrophilic group on the first polymer may comprise a sulfonyl group that reacts with a reactive nucleophilic group on the second polymer comprising an amino group to at least partially covalently link the first polymer to the second polymer. The second reactive electrophilic group on the first polymer may comprise an amino group, thiol group, or hydroxyl group, for example to covalently bond or link the first polymer to the existing layer on the lens core

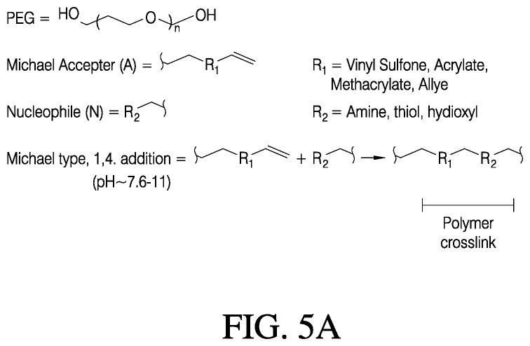

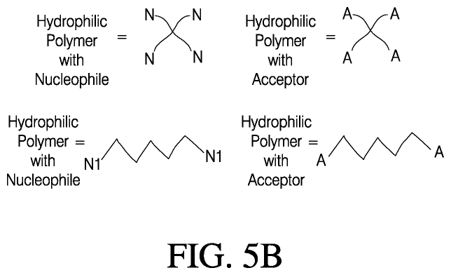

In some embodiments, the reaction includes cross-linking mechanisms and/or other types of reactions between a first and second polymer species or between different reactive groups in a population of a polymer species. For example, the first and second hydrophilic polymers or the first reactive group and the second reactive group react with a hydrophilic hydrogel coated surface of the medical device as well as with each other to form or renew the hydrophilic hydrogel layer that is at least partially covalently crosslinked to the medical device. For example, the reaction may include any number of suitable methods known in the art including photochemical or thermal cross-linking. In some cases, cross-linking may occur through nucleophilic conjugate reaction, Michael-type reaction (e.g. 1,4 addition), and/or Click reaction between respective reactive groups on more than one polymer species in the hydrophilic layer. FIG. 5A shows an exemplary Michael-type reaction between a Michael Acceptor (A) (e.g., vinyl sulfone, acrylate, methacrylate, allyl, etc.) and a nucleophile (N) (e g, amine, thiol, hydroxyl, etc.). Michael-type 1,4 addition of the acceptor and nucleophile results in a linkage or bond. For example, a vinyl sulfone group reacts with an amine to form a sulfonamide linkage or bond; a carboxyl group and an amine group react to form an amide linkage or bond; and a thiol group and vinyl sulfone group react to form a thioether linkage or bond.

In some embodiments, the reactive group(s) (e.g., sulfonyl, amine, carboxyl, thiol, hydroxyl, etc.) in the regenerating or rejuvenating solution react with the remaining or available reactive groups (e.g., sulfonyl, amine, carboxyl, thiol, hydroxyl, etc.) on the surface of the coated medical device. For example, there may be available or remaining pendant or terminal or otherwise present groups that are accessible for bonding to the reactive group(s) in the regenerating or rejuvenating solution. Alternatively or additionally, the polymers and/or reactive groups in the solution may become physically entangled with the existing hydrogel coating on the medical device, and when the reactive groups on the polymers in the regenerating solution react with each other, permanent physical entanglements or covalent linkages are formed. Alternatively or additionally, in some embodiments, one or more reactive groups (e.g., carboxyl, hydroxyl, amine, etc.) on a surface of the lens core are available for bonding with the reactive groups present in the regenerating or rejuvenating solution.

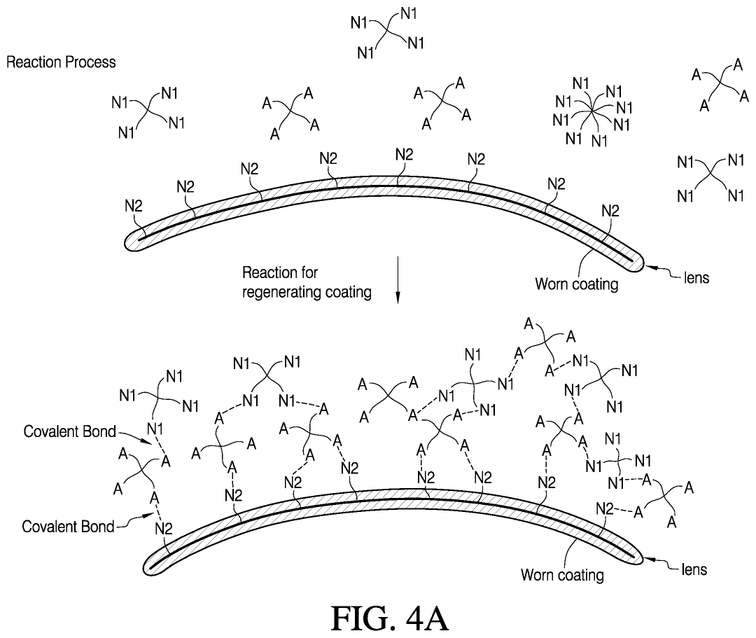

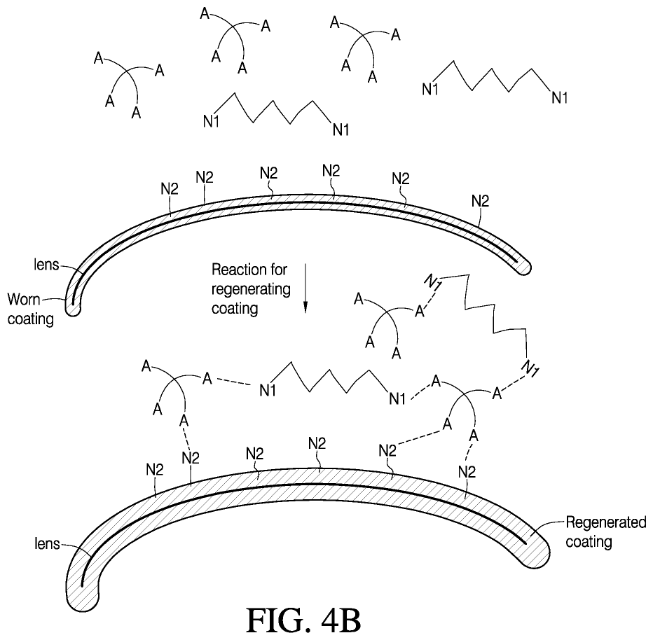

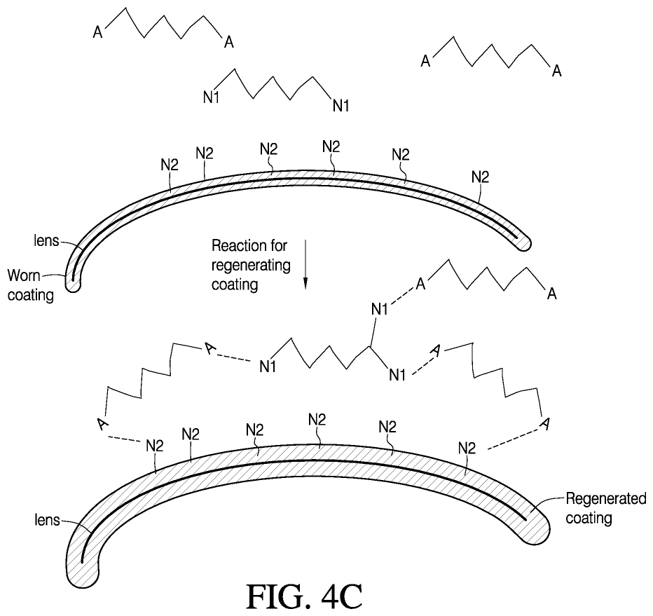

In some embodiments, as shown in FIGS. 4A-4C and 5A-5B, a medical device or lens to be rejuvenated or refreshed by the solutions and methods described herein may include or have a coating thereon. The original or existing coating may comprise a water content of greater than 80%, greater than 90%, greater than 95%, greater than 98%, 80-99%, 85-95%, 90-98%, 95-99%. The original or existing coating of any of the preceding embodiments may have an advancing contact angle of 20-35 degrees, 25-30 degrees, 30-35 degrees, 25-35 degrees, etc. The original or existing coating of any of the preceding embodiments may be substantially free of silicone; may consist of 0% silicone; may consist of 0% silicone; may include less than 5% silicone, less than 2% silicone, less than 1% silicone; may include 0-5% silicone, 0-3% silicone, etc. In any of the preceding embodiments, the original coating may comprise PEG and polyacrylamide polymers; PEG and PEG polymers; or polyacrylamide and polyacrylamide polymers.

The original or existing coating may be a new coating or a worn or reduced coating. Such worn or reduced coating may be worn as a result of wear or use by a user, degraded as a result of environmental conditions, or diminished from rubbing, soaking, or any other activity or use that may degrade or reduce the coating. In some embodiments, a worn coating has a reduced thickness compared to a fresh or new coating or recent coating. For example, a new or fresh coating may have a thickness of around or substantially 50 nm in thickness, 1 nm to 50 nm in thickness, less than 50 nm in thickness, or 50 nm to 100 nm in thickness. In contrast, a worn coating may have a thickness of less than 1 nm, less than 5 nm, less than 10 nm, less than 15 nm, less than 20 nm, less than 25 nm, less than 30 nm, less than 35 nm, less than 40 nm, or less than 45 nm. In some embodiments, a worn coating may have a thickness of 0.001 to 10 nm, 10 to 20 nm, 20 to 30 nm, 30 to 40 nm, 40 to 50 nm, or any range or subrange therebetween. In some embodiments, a worn coating is 1-10%, 10-20%, 20-30%, 30-40%, 40-50%, 50-60%, 60-70%, 70-80%, 80-90%, or 90-100% less than the original coating thickness. In some embodiments, the worn coating is rejuvenated or refreshed to 1-10%, 10-20%, 20-30%, 30-40%, 40-50%, 50-60%, 60-70%, 70-80%, 80-90%, 90-100%, 100-150%, 150-200%, 200-250%, 250-300%, 300-350%, 350-400%, 400-450%, 450-500%, or any range or subrange therebetween of the thickness of the original coating.

As shown in FIGS. 4A and 5B, the refreshed or rejuvenated hydrophilic polymer layer includes a first branched polymer species having a reactive group A and second branched polymer species with a reactive group N1. A branched species may include a polymer having a branch count ranging from 2-arm to 12--arm branching. In other embodiments, the branched species may include starred branching with about 100 branches or more. The hydrophilic polymer layer may be formed by cross-linking the first polymer species and the second polymer species through a reaction between reactive group A and N1. In some embodiments, the worn coating includes a reactive moiety N2. Reactive moiety N2 may be adapted to react with reactive groups of polymer species in the hydrophilic polymer layer. In some cases, the reactive moiety N2 only reacts with one of the polymer species, for example A or N1. Reactive moiety N2 reacts with reactive group A on the first species to form a covalent attachment between the hydrophilic polymer layer and the worn coating. Reactive moiety N2 may already be present on a surface of the worn coating or may be revealed or otherwise made available during a cleaning process (to remove debris, dirt, proteins, etc.). In some embodiments, reactive moiety N2 is revealed by surface activation, as described elsewhere herein. As shown in FIG. 4A, cross-linkages covalently link the first and second polymers to form a hydrophilic polymer layer on a worn hydrogel layer on a surface of the medical device. In some embodiments, the hydrogel layer coats an entire surface, surrounds an entire surface, coats a first side, coats a second side, coats a convex surface, coats a concave surface, and/or coats any or all surfaces of a medical device, for example a contact lens, as shown in FIG. 4A.

FIG. 4B is similar to FIG. 4A except that one or more of the polymer species in the rejuvenating solution may be branched or a chain polymer with one or more pendant reactive groups, as shown in FIG. 5B. Similarly, FIG. 4C is similar to FIGS. 4A-4B except that both of the polymer species in the rejuvenating solution may be a chain polymer with one or more pendant reactive groups, as shown in FIG. 5B. One or more of the polymers may additionally or alternatively include a cyclic or heterocyclic ring therein. The branching feature of each polymer may impact an overall steric hindrance between polymers and/or between the polymers and reactive groups in the original coating, alter a molecular weight of the polymer, etc. which may affect a compaction of the rejuvenated coating on the lens or an efficiency of the reaction in forming the rejuvenated coating, and/or alter a wettability, lubricity, or other characteristic of the rejuvenated lens.

In some embodiments, the reaction of the first and second hydrophilic polymers with the surface of the lens or with a worn coating existing on the lens forms a hydrophilic hydrogel layer to reduce the wetting angle of the contact lens.

As described elsewhere herein, a medical device that may be used in a rejuvenation process or in a rejuvenation method may include an existing hydrophilic polymer layer on a surface of the medical device. The existing hydrophilic polymer layer may be covalently linked to the surface of the medical device through one or more functional groups, for example amines, amino-reactive groups, sulfhydryl, sulfhydryl-reactive groups, carboxyl groups, hydroxyl groups, haloalkyl groups, dienophile groups, aldehyde or ketone groups, alkenes, epoxides, phosphoramidites, or a combination thereof.

In some embodiments, recoating, retreating, or rejuvenating a coated medical device does not require activation of the coated surface before recoating or rejuvenating the existing coating. For example, the surface does not need to be activated using gas plasma, light activation, activation of a liquid monomer mix, wet activation (e.g., with IPA, ozone, other chemical, etc.), and/or adding a monomer that reacts with the contact lens that still leaves reactive sites. As such, a surface of the coated medical device is simply cleaned (to remove proteins, lipids, and/or dirt), and then the existing coating is rejuvenated or recoated using the methods described elsewhere herein.

Alternatively, in some embodiments, an existing coated surface of a medical device requires activation before recoating or rejuvenating the existing coating. For example, a type of material or a composite of materials comprising the medical device or the existing coating on the medical device determine the type of activation (e.g., gas plasma, light activation, activation of a liquid monomer mix, wet activation, and/or adding a monomer that reacts with the contact lens that still leaves reactive sites). For example, a type of material or a composite of materials comprising the medical device or the existing coating on the medical device determines a type of gas required for surface activation by gas plasma. For example, the gas may be oxygen, helium, argon, ammonia, n-heptylamine, nitrogen, carbon dioxide, hydrogen, tetrafluoromethane, sulfur tetrafluoride, octafluorocyclobutane, air, or another type of gas. Various types of gases and their uses are described, for example, in Hall et al. “Activated gas plasma surface treatment of polymers for adhesive bonding.” J Appl Polymer Sci. 1969; 13(10), Pp. 2085-2096; Guruvenket et al. “Plasma surface modification of polystyrene and polyethylene.” Appl Surf Sci. 2004; 236(1-4), pp 278-284.; Liston et al. “Plasma surface modification of polymers for improved adhesion: a critical review.” J Adhesion Sci and Tech. 1993; 7(10), pp. 1091-1127, each of which are herein incorporated by reference in their entireties. The type of gas used in the surface activation of the medical device may determine which chemical groups are present on the polymers reacting in the reaction to produce a hydrogel polymer coating on the surface of the medical device or rejuvenate an existing coating on the medical device. Gases such as carbon dioxide, oxygen, nitrogen, ammonium, hydrogen, or their blends may be used when reactive groups such as hydroxyl, carboxyl, and/or amine are desired on the surface or existing coating. Argon or helium gases may be used to produce radicals that will react with oxygen or water in the air to form peroxides, which will be able to initiate further grafted polymerization of monomers. The type of gas used in the activation process may further depend on various processing requirements for the medical device, for example ability to be exposed to vacuum, ability to be exposed to high temperatures, etc. Thus, depending on the desired functional groups in the reaction and a type of medical device, a desirable gas or combination of gases may be selected for plasma activation of the surface of the medical device.

In some embodiments, a type of medical device and/or a wear or use profile of the medical device determines a desired thickness of the hydrogel polymer layer on the medical device after rejuvenating or regenerating the coating. For example, an initial thickness (before use or wear) and/or a final thickness (after rejuvenating or replenishing) of the hydrogel polymer layer on the medical device may be 0 to 50 nm; 0 to 100 nm; 50 nm to 100 nm; 0 to 250 nm; 0 to 500 nm, 0 to 1 micron, 0.5 to 1 micron, 1 to 5 micron, or any range or subrange therebetween. A thickness of the rejuvenated coating may be controlled by a molar ratio of reactants or chemical groups, a molarity of reactants or chemical groups, a pH of the reaction, a temperature of the reaction, a degree or complexity of electrostatic interactions in the reaction, a degree or complexity of steric hindrance in the reaction, a molecular weight of reactants or chemical groups, etc.

In one non-limiting embodiment, a weight ratio between a first polymer and a second polymer is 0.2-0.6 g of the first polymer to 0.8-1.2 g of the second polymer. Alternatively, the weight ratio of a first polymer to a second polymer may be 0.1-0.5 g to 0.5-1.1 g; 0.2-0.5 g to 0.7-1.1 g; 0.3-0.5 g to 0.9-1.1 g; 0.1-1 g to 0.5-1.5 g, etc. In another non-limiting embodiment, a reactive group ratio between a first polymer and a second polymer is 2-4 reactive groups to 1-3 reactive groups. Alternatively, the reactive group ratio of the first polymer to the second polymer may be 1-5 to 1-4; 1-10 to 1-8; 3-9 to 1-7, etc.

In some embodiments, a second polymer is in molar excess in the solution compared to the first polymer, for example 1-5 times in excess, 2-3 times in excess, 2-8 times in excess, 1-6 times in excess, etc. For example, in a solution comprising PEG and polyacrylamide, polyacrylamide may be in molar excess of PEG. In some embodiments, an electrophilic group may be in molar excess of a nucleophilic group, for example 0.5-2 times in excess, 1-2 times in excess, 1-5 times in excess, 0.5-4 times in excess, etc. For example, in a solution comprising a sulfonyl moiety and an amine moiety, the sulfonyl moiety may be in molar excess of the amine moiety.

In some embodiments, the reaction is configured to produce a hydrogel coating on a surface of a medical device that has a higher (e.g., 80 to 99%) water content or a lower water content (50 to 80%) depending on a type of the medical device.

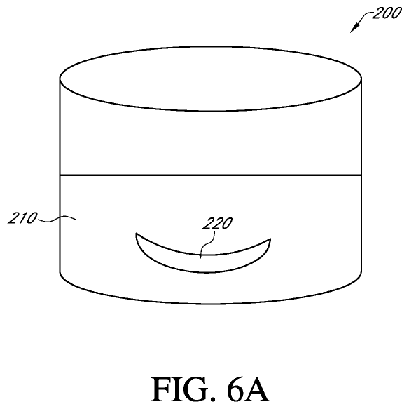

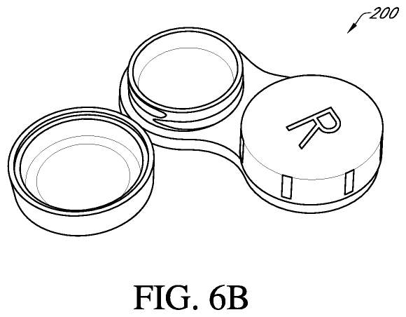

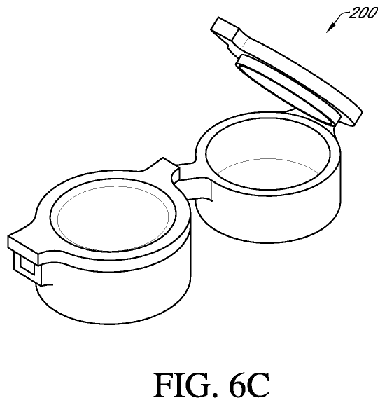

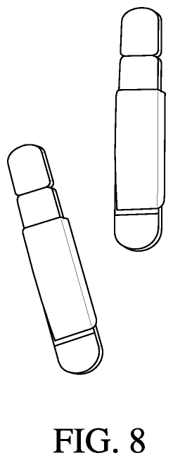

Another aspect of the present disclosure is directed to a kit for rejuvenating or refreshing an existing or current coating on a medical device or a previously coated medical device, as shown in FIGS. 6A-8. In some embodiments, as shown in FIGS. 6A-6C and 8, the kit includes a receptacle containing a hydrophilic polymer solution comprising a first and second polymer. In some embodiments, the kit includes a first receptacle containing a first polymer having one or more reactive functionalities; and a second receptacle containing a second polymer having one or more reactive functionalities. The kit may optionally further include a case defining at least one opening to allow a medical device, e.g., contact lens, therethrough, such that the case is configured to hold the contents of the first and second receptacles during an incubation period. Alternatively or additionally, one or more of the receptacles defines at least one opening to allow a medical device therethrough, the receptacle also being configured to contain a solution therein.

In some embodiments, the first and second polymers are configured to react in a receptacle or case or otherwise to form an at least partially covalently crosslinked hydrogel coating between the first and second polymers and an existing coating on the medical device. The medical device may have been previously coated with a hydrogel coating and worn, such that an original or initial thickness of the hydrogel coating on the medical device is reduced to a second thickness.

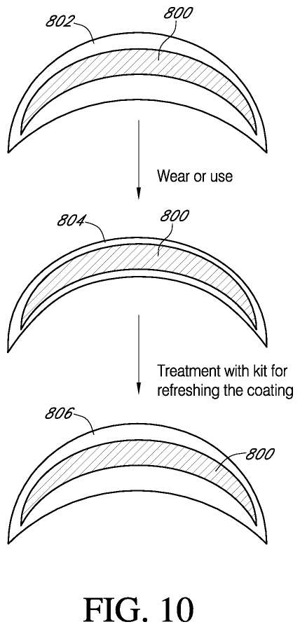

In some embodiments, treating or coating or refreshing a medical device comprises treating or retreating the medical device to regenerate a coating, build upon an existing coating, add to an existing coating, or create a second coating on top of the existing coating, for example a previously applied coating (e.g., a hydrogel layer or coating). As shown in FIG. 10, the original coating on the medical device 800 has a first thickness or baseline thickness 802 that is worn down to a second thickness 804 or a wear-based thickness or reduced thickness, the second thickness 804 being less than the first or baseline thickness 802. Using one or more solutions, methods, or kits described herein, the original coating having the second thickness 804 on the medical device, when treated with a rejuvenating or refreshing solution, kit, or system, is regenerated substantially back to a baseline or a first thickness, shown as final thickness 806. In some such embodiments, substantially refers to 80% to 130%, 90% to 115%, 90% to 125%, 100% to 110%, 95% to 110%, etc. of the baseline or first thickness. In some embodiments, the regenerated or refreshed or rejuvenated coating is substantially uniform in thickness. In other embodiments, the regenerated or refreshed or rejuvenated coating is substantially uniform in thickness on a first side and/or a second side or a first surface and/or a second surface.

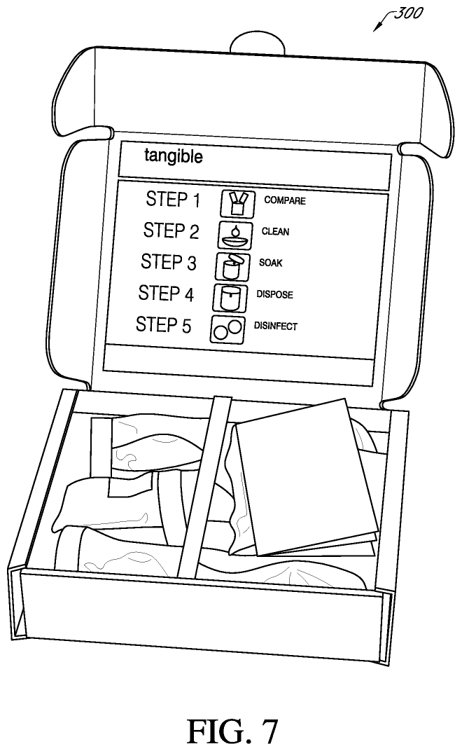

The solution may be provided as part of a kit 300 for treating or refreshing contact lenses, for example lenses with a worn coating, as shown in FIG. 7. The kit 300 may comprise a first receptacle comprising a first polymer solution; a second receptacle comprising a second polymer solution; and optionally, a case comprising at least one opening to allow a contact lens therethrough. Alternatively, in some embodiments, the kit 300 may include one receptacle comprising the first and second polymer solutions therein. In some embodiments, the case or one or more receptacles is configured to hold a solution comprising one or more hydrophilic polymers for at least 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100 minutes, 1 to 3 days, 24 hours, 48 hours, etc. or any range or subrange therebetween. The first and second polymers are configured to react in the case or receptacle or otherwise to form a hydrogel coating on the medical device, for example a contact lens, the medical device either having an existing coating or no existing coating. In some embodiments, the reaction between the first polymer solution and the second polymer solution occurs at room temperature, for example 20 to 40° C., 20 to 30° C., 10 to 40° C., 15 to 40° C., 20 to 25° C., etc.

In some embodiments, the kit 300 may include additional components such as instructions for use, a contact lens case, and/or other cleaning or disinfecting solutions. Additionally, the kit may package the components singularly or in various combinations, for example, with the first polymer and the second polymer each within a separate foil pouch or other receptacle or combined together, ready for use. Additionally, the kit may use visual, tactile, or auditory aids to improve user adherence to various methods of use of the kit. In some embodiments, the receptacle or case in which the contact lens is treated may be clear, translucent, or opaque, and it may include a reversibly sealable cap. The sealable cap may additionally or alternatively be configured such that it holds the contact lens so that the contact lens is immersed in the solution when the cap is closed.

As an additional example, as shown in FIG. 8, the kit may include a first and second receptacle, as described elsewhere herein, for each of the different polymers. In some embodiments, color coding or differing shapes is used to distinguish a first vial from a second vial. For example, a blue vial indicates a first polymer and an orange vial indicates a second polymer. Such vials may take any shape, structure, and/or color, and in the present example, are shown as tubes.

As shown in FIGS. 6A-6C, a kit for rejuvenating or refreshing coated medical device may include a case or receptacle 200 in which the medical device is treated. The case 200 may be of any suitable shape or structure. For example, the case 200 may be barrel-shaped (FIG. 6A), disc-shaped (FIG. 6B), cylindrical-shaped (FIG. 6C), oval-shaped, or any other conceivable shape. In some embodiments, the case includes a sealable opening, such that the seal is formed of adhesive, a threaded cap (FIG. 6B), snap-fit connection (FIG. 6C), magnets, or any other type of sealing mechanism.

As shown in FIG. 6A, solution 210 is in a receptacle or case 200, which also contains the medical device, for example a contact lens 220. In some embodiments, a first polymer solutions 101 and a second polymer solutions 102 are poured into or added to receptacle 200, and the contact lens 220 is thereafter added to the resulting solution 210. Optionally, however, any sequence may also be envisioned, which leads to the end result of the lens in the solution containing the first and second polymers. For example, the first polymer solution and the lens may be initially added to the receptacle, then the second polymer solution can be added to the receptacle to mix the first and second solutions. Alternatively, the first and second polymer solutions may be included in the kit in a pre-mixed solution, such that the medical device only needs to be added to the solution.

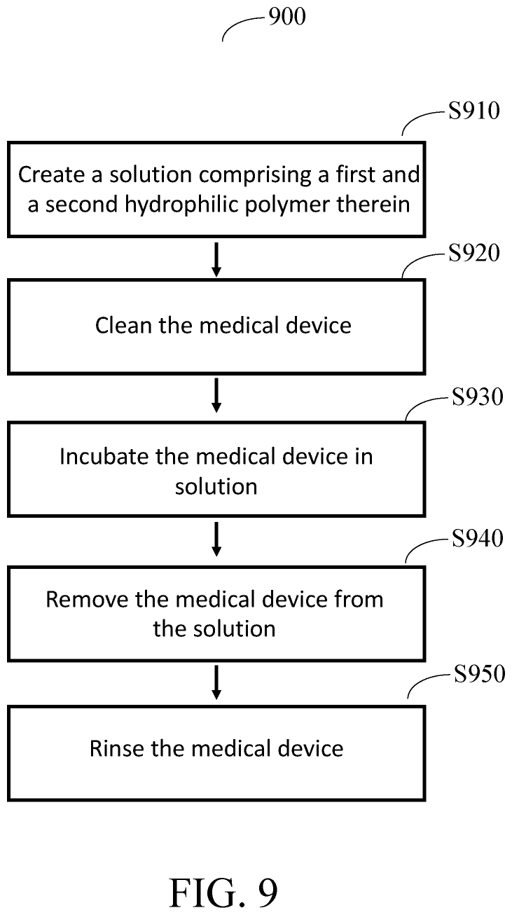

FIG. 9 shows one embodiment of a method 900 of using a kit for treating or refreshing a medical device. Block S910 recites: create or provide a solution comprising a first hydrophilic polymer and a second hydrophilic polymer. In some embodiments, the first and second hydrophilic polymers are pre-combined in the kit for the user; in other embodiments, the first and second hydrophilic polymer solutions require mixing before use. Block S920 recites: clean the medical device. In some embodiments, the solution from block S910 is allowed to rest while the user cleans the medical device prior to treatment. Cleaning may comprise rubbing the medical device with a cleaning or multipurpose solution prior to treatment with the solution in block S610. In some embodiments, cleaning functions to remove protein build-up or other debris (e.g., from use) from a surface of the medical device; in other embodiments, cleaning functions to remove an original coating on the medical device. In some embodiments, cleaning functions to activate an existing coating on the medical device, as described elsewhere herein. Alternatively, the cleaning does not activate an existing coating such that there are already available reactive groups for reacting with the first and second polymers in the rejuvenating solution. Block S930 recites: incubate medical device in the solution. As shown elsewhere herein, the cleaned medical device from block S920 is soaked in a receptacle with a solution from block S910. In some embodiments, the medical device is stored within a protrusion on the inside of the cap of the receptacle, such that once the cap is sealed, the protrusion allows the medical device to come into contact with the solution. In other embodiments, the medical device floats or is otherwise untethered or suspended while incubating in the solution. In still other embodiments, the medical device is dipped once or repeatedly in the solution or sprayed with the solution to coat the medical device. The medical device may be treated in the solution at room temperature, at refrigeration temperatures, at freezer temperatures, or in a heated solution (e.g., heated by boiling, microwaving, etc.).

In some embodiments, one treatment session includes one incubation period, more than one incubation period, or a plurality of incubation periods. The incubation period comprises the time needed to refresh a coating on the medical device or create a new coating on the medical device. In some embodiments, the incubation period is the time needed for the reaction between the first and second hydrophilic polymer solutions to reach equilibrium. The medical device may be treated daily, weekly, monthly, quarterly, biannually, or yearly to increase or maintain or regenerate the coating on the medical device. An incubation period may comprise 0-10 minutes, 10-20 minutes, 20-30 minutes, 30-40 minutes, 40-50 minutes, 50-60 minutes, or any range or subrange therebetween. An incubation period may comprise at least 10 minutes, at least 20 minutes, at least 30 minutes, at least 40 minutes, at least 50 minutes, or at least 60 minutes. In some embodiments, an incubation period increases or decreases based on a temperature of the solution.

In some embodiments, a frequency of treatment may be patient-dependent or a frequency of treatment may be recommended. In some embodiments, a resulting final or regenerated thickness may be independent of a wear-based thickness or a thickness that results after wear and/or use, such that the solutions and methods described herein are configured to generate a substantially fixed final thickness regardless of a starting thickness of the original coating. The fixed final thickness may be a result of known molar ratios of reactive groups and/or polymers in the rejuvenating solution. For example, the required coating thickness to maintain wettability is likely dependent on a roughness of the substrate. For hard contact lenses (e.g., RGP lenses), the initial coating thickness is typically about or substantially 30-40 nm but may be 0 to 50 nm, less than 50 nm, or 25 nm to 50 nm, and the wettability declines significantly when about half of the coating has worn away. During use and/or wear, the coating thickness tends to plateau, so regenerating or rejuvenating treatments, as described elsewhere herein, on a thicker original coating do not add as many nanometers to the thickness as the regenerating or rejuvenating treatment performed on a thinner coating. In some embodiments, increasing the polymer concentrations or concentrations of reactive groups may increase the recoating thickness.

Block S940 recites: remove the medical device from the solution. In some embodiments, the solution may be disposed of or reused once, more than once, or a plurality of times. Block S950 recites: rinse the medical device. The medical device may be rinsed or disinfected in a cleaning solution or a multipurpose solution for an hour, several hours (e.g., overnight), a day, a week, or at least prior to use.

Various embodiments of the inventions will now be described herein below.

In one exemplary embodiment, a solution for regenerating a coating on a surface of a hydrogel coated medical device, wherein an initial thickness of an original hydrogel coating is greater than a second thickness of the original hydrogel coating after use or wear, comprises: a polyethylene glycol polymer species comprising 2 to 12 branch arms, wherein one or more of the branch arms comprise a terminal sulfonyl reactive group; and a polyacrylamide polymer species comprising one or more pendant amine groups along a backbone of the polyacrylamide polymer species, wherein the PEG and polyacrylamide polymer species are adapted to covalently bond with one or more available reactive groups in the original hydrogel coating having the second thickness as well as with each other to increase the second thickness of the original hydrogel coating on the medical device so that a final thickness of the regenerated coating approaches or exceeds the initial thickness.

In any of the preceding embodiments, the final thickness of the regenerated coating is substantially equal to the initial thickness of the original hydrogel coating.

In any of the preceding embodiments, the original hydrogel coating on the medical device does not require activation prior to covalently bonding the PEG and polyacrylamide species to the original hydrogel coating.

In any of the preceding embodiments, the first and second hydrophilic polymers are adapted to regenerate or refresh the hydrogel coating on the medical device.

In any of the preceding embodiments, the original hydrogel coating is formed by covalently bonding a first polymer species and a second polymer species to the surface of the medical device.

In any of the preceding embodiments, the solution further comprises a buffering agent.

In any of the preceding embodiments, the buffering agent is triethanolamine.

In any of the preceding embodiments, the medical device is a contact lens.

In any of the preceding embodiments, the medical device is configured to be implantable within a mammalian body.

In any of the preceding embodiments, the medical device is a stent configured to keep a cavity open.

In any of the preceding embodiments, the stent is configured to keep a blood vessel, bile duct, intestine, nasal passage or cavity, sinus cavity, or intraocular channel open.

In any of the preceding embodiments, the medical device is a sensor, camera, vital sign monitor, drug depot device, neurostimulator, ultrasound, silicone implant, saline implant, hernia mesh, penile implant, orthopedic rod or plate or pin or nails, pacemaker, cardiac valve, ear tube, aneurysm coil, or intraocular lens.

In any of the preceding embodiments, the medical device is a test strip.

In any of the preceding embodiments, the medical device is a drug, salivary, urine, blood or semen test strip.

In any of the preceding embodiments, the medical device is a tool configured to be inserted within a mammalian body.

In any of the preceding embodiments, the medical device is a catheter, trocar, endoscope, or laparoscope.

In any of the preceding embodiments, the medical device is configured to be used externally on a mammalian body.

In any of the preceding embodiments, the medical device is configured for use as a bandage, wound dressing, external sensor, hearing aid, or artificial skin.

In any of the preceding embodiments, an outer surface of the medical device comprises one or more of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, or silk.

In any of the preceding embodiments, an outer surface of the device consists essentially of a material selected from the group consisting of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, or silk.

In any of the preceding embodiments, a pH of the solution is between 8 and 11.

In any of the preceding embodiments, the regenerated coating on the contact lens reduces a wetting angle of the contact lens.

In any of the preceding embodiments, the original hydrogel coating comprises an amino-reactive group, a sulfonyl reactive group, or a combination thereof.

In any of the preceding embodiments, the original hydrogel coating comprises a water content of greater than 90%.

In any of the preceding embodiments, the original hydrogel coating has an advancing contact angle of 20-35 degrees.

In any of the preceding embodiments, the original coating is substantially free of silicone.

In any of the preceding embodiments, the original coating consists of less than 2% silicone.

In any of the preceding embodiments, the original coating comprises less than 2% silicone.

In any of the preceding embodiments, the original coating comprises PEG and polyacrylamide polymers; PEG and PEG polymers; polyacrylamide and polyacrylamide polymers; or a combination thereof.

In one exemplary embodiment, a kit for treating contact lenses comprises: a first receptacle configured to reversibly retain a first solution therein, wherein the first solution comprises a polyethylene glycol (PEG) polymer species comprising 2 to 12 branch arms, wherein one or more of the branch arms comprise a terminal sulfonyl reactive group; a second receptacle configured to reversibly retain a second solution therein, wherein the second solution comprises a polyacrylamide polymer species comprising one or more pendant amine groups along a backbone of the polyacrylamide polymer species; and a case comprising at least one opening to allow a medical device through, wherein the case is configured to retain the contents of the first and second receptacles for at least 30 minutes.

In any of the preceding embodiments, the PEG and polyacrylamide polymer species are adapted to covalently bond with one or more available reactive groups in the original hydrogel coating having the second thickness as well as with each other to increase the second thickness of the original hydrogel coating on the medical device so that a final thickness of the regenerated coating approaches or exceeds the initial thickness.

In any of the preceding embodiments, the first and second polymers are configured to react in the case to form an at least partially covalently crosslinked hydrogel coating on the medical device.

In any of the preceding embodiments, the kit further comprises the medical device, wherein the medical device comprises a hydrogel layer on a surface of the medical device.

In any of the preceding embodiments, the hydrogel layer is covalently bound to the surface of the medical device and comprises a first polymer species and a second polymer species that are at least partially covalently cross-linked.

In any of the preceding embodiments, the hydrogel layer on the surface of the medical device comprises an initial thickness before use or wear and a second thickness after use or wear, the second thickness less than the initial thickness.

In any of the preceding embodiments, the first and second solutions are reacted in the case to increase the second thickness to yield a final thickness that approaches, is substantially equal to, or exceeds the initial thickness.

In one exemplary embodiment, a method of treating a medical device to form a hydrogel coating thereon comprises: providing a medical device having a hydrogel layer covalently attached to at least a portion of an outer surface of the medical device, wherein the hydrogel layer comprises a hydrophilic polymer population having a first species and a second species, the first species being at least partially cross-linked to the second species, wherein the first species comprises a reactive electron pair accepting group and the second species comprises a reactive nucleophilic group, the reactive electron pair accepting group and the reactive nucleophilic group adapted to react to thereby form cross-links between the first species and the second species, wherein the hydrogel layer has an initial thickness before use or wear and a second thickness after use, the initial thickness being greater than the second thickness; removing protein and lipid deposits from the medical device having the hydrogel layer having the second thickness after use or wear, wherein removing protein and lipid deposits from the medical device does not activate the outer surface of the medical device; creating or providing a mixed solution comprising a first solution comprising a first polymer and a second solution comprising a second polymer; incubating the medical device in the mixed solution at room temperature for a predetermined length of time; and rinsing the medical device.

In any of the preceding embodiments, the mixed solution reacts with the hydrogel layer to thereby form crosslinks and increases the second thickness so that it substantially approaches, equals, or exceeds the initial thickness.

In any of the preceding embodiments, the initial thickness is 25 nm to 100 nm.

In any of the preceding embodiments, the second thickness is 10 nm to 80 nm.

In any of the preceding embodiments, the predetermined length of time is 15 to 45 minutes.

In any of the preceding embodiments, the predetermined length of time is 30 minutes.

In any of the preceding embodiments, the incubating step occurs between 20 to 40° C.

In any of the preceding embodiments, the incubating step occurs between 20 to 25° C.

In any of the preceding embodiments, the initial thickness is between 20 nm and 100 nm.

In any of the preceding embodiments, the second thickness is between 5 nm and 80 nm.

In any of the preceding embodiments, the initial thickness is between 30 nm and 75 nm.

In any of the preceding embodiments, the second thickness is between 5 nm and 60 nm.

In any of the preceding embodiments, the first species is PEG and the second species is polyacrylamide.

In any of the preceding embodiments, the first species is polyacrylamide and the second species is polyacrylamide.

In any of the preceding embodiments, the reactive electrophilic group is selected from the group consisting of amino-reactive groups, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the reactive nucleophilic group is selected from the group consisting of amines, amino-reactive groups, sulfhydryl, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the method further includes covalently attaching the original hydrogel layer on the medical device.

In any of the preceding embodiments, the method further includes forming the original hydrogel layer on the medical device.

In any of the preceding embodiments, forming the original hydrogel layer includes: reacting an outer surface of the medical device with a first polymer species of a hydrophilic polymer solution, wherein the first polymer species comprises a moiety at a first portion that forms a covalent attachment to the outer surface of the device; and reacting the first polymer species of the hydrophilic polymer solution with a second polymer species of the hydrophilic polymer solution, the second polymer species comprising a moiety that forms a covalent bond to a second portion of the first polymer species in a second covalent reaction thereby forming the original hydrogel coating comprising the first polymer species and the second polymer species at least partially cross-linked.

In any of the preceding embodiments, the moiety at the first portion, the second portion, or both of the first polymer species comprises a reactive sulfonyl group.

In any of the preceding embodiments, the moiety of the second polymer species comprises a reactive amino group.

In any of the preceding embodiments, the first polymer species comprises PEG or polyacrylamide.

In any of the preceding embodiments, the second polymer species comprises PEG or polyacrylamide.

In any of the preceding embodiments, the moiety at the first portion, the second portion, or both of the first polymer species comprises a reactive carboxyl group.

In any of the preceding embodiments, the moiety of the second polymer species comprises a reactive amino group.

In one exemplary embodiment, a method of treating a medical device to form a hydrogel coating thereon comprises: providing a medical device having a hydrogel layer covalently attached to at least a portion of an outer surface of the medical device, wherein the hydrogel layer comprises a hydrophilic polymer population having a PEG species and a polyacrylamide species, the PEG species being at least partially cross-linked to the polyacrylamide species, wherein the PEG species comprises a reactive electron pair accepting group and the polyacrylamide species comprises a reactive nucleophilic group, the reactive electron pair accepting group and the reactive nucleophilic group adapted to react to thereby form cross-links between the PEG species and the polyacrylamide species, wherein the hydrogel layer has an initial thickness before use and a second thickness after use, the initial thickness being greater than the second thickness; removing protein and lipid deposits from the medical device having the hydrogel layer having the second thickness after use, wherein removing protein and lipid deposits from the medical device does not activate the outer surface of the medical device; creating or providing a mixed solution comprising a first solution comprising a PEG polymer and a second solution comprising a polyacrylamide polymer; incubating the medical device in the mixed solution at room temperature for a predetermined length of time, wherein the mixed solution reacts with the hydrogel layer to thereby form crosslinks and increase the second thickness so that is approaches or exceeds or is substantially equal to the initial thickness; and rinsing the medical device.

In any of the preceding embodiments, the predetermined length of time is 1 to 30 minutes.

In any of the preceding embodiments, the predetermined length of time is at least 30 minutes.

In one exemplary embodiments, a solution for regenerating a coating on a surface of a hydrogel coated medical device, wherein an initial thickness of an original hydrogel coating is greater than a second thickness of the original hydrogel coating after use or wear, comprises: a first polymer species comprising a reactive electrophilic; and a second polymer species comprising a nucleophilic group complementary in reactivity to the reactive electrophilic group, wherein the first and second polymer species are adapted to covalently bond with one or more available reactive groups in the original hydrogel coating having the second thickness as well as with each other to increase the second thickness of the original hydrogel coating on the medical device so that a final thickness of the regenerated coating approaches the initial thickness, wherein the final thickness of the regenerated coating is substantially equal to the initial thickness of the original hydrogel coating.

In any of the preceding embodiments, the original hydrogel coating on the medical device does not require activation prior to covalently bonding the first and second species to the original hydrogel coating.

In any of the preceding embodiments, the first and second polymers are adapted to regenerate or refresh the hydrogel coating on the medical device.

In any of the preceding embodiments, the original hydrogel coating is formed by covalently bonding a first hydrophilic polymer and a second hydrophilic polymer to the surface of the medical device.

In any of the preceding embodiments, the first polymer species is polyethylene glycol (PEG) and the second polymer species is polyacrylamide.

In any of the preceding embodiments, the first polymer species is polyacrylamide and the second polymer species is polyacrylamide.

In any of the preceding embodiments, the reactive electrophilic group is selected from the group consisting of amino-reactive groups, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the reactive nucleophilic group is selected from the group consisting of amines, amino-reactive groups, sulfhydryl, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the medical device is a contact lens.

In any of the preceding embodiments, the medical device is configured to be implantable within a mammalian body.

In any of the preceding embodiments, the medical device is a stent configured to keep a cavity open.

In any of the preceding embodiments, the stent is configured to keep a blood vessel, bile duct, intestine, nasal passage or cavity, sinus cavity, or intraocular channel open.

In any of the preceding embodiments, the medical device is a sensor, camera, vital sign monitor, drug depot device, neurostimulator, ultrasound, silicone implant, saline implant, hernia mesh, penile implant, orthopedic rod or plate or pin or nails, pacemaker, cardiac valve, ear tube, aneurysm coil, or intraocular lens.

In any of the preceding embodiments, the medical device is a test strip.

In any of the preceding embodiments, the medical device is a drug, salivary, urine, blood or semen test strip.

In any of the preceding embodiments, the medical device is a tool configured to be inserted within a mammalian body.

In any of the preceding embodiments, the medical device is a catheter, trocar, endoscope, or laparoscope.

In any of the preceding embodiments, the medical device is configured to be used externally on a mammalian body.

In any of the preceding embodiments, the medical device is configured for use as a bandage, wound dressing, external sensor, hearing aid, or artificial skin.

In any of the preceding embodiments, an outer surface of the medical device comprises one or more of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, or silk.

In any of the preceding embodiments, an outer surface of the device consists essentially of a material selected from the group consisting of: glass, plastic, titanium, nitinol, polyethylene, polypropylene, polyvinyl chloride, polytetraflouroethylene, polydimethylsiloxane, polyethylene terephthalate, polyamides, polyether urethane, polyether urethane urea, polystyrene, polycarbonate, polysulfones, polymethyl methacrylate, poly 2-hydroxyethylmethacrylate, polyvinylalcohol, polyglycolic acid, polycaprolactone, polylactic acid, polyortho ester, cellulose acetate, collagen, or silk.

In any of the preceding embodiments, a pH of the solution is between 8 and 11.

In any of the preceding embodiments, the regenerated coating on the contact lens reduces a wetting angle of the contact lens.

In any of the preceding embodiments, the final thickness of the regenerated coating is substantially equal to the initial thickness of the original hydrogel coating

In one exemplary embodiments, a kit for treating contact lenses comprises: a first receptacle configured to reversibly retain a first solution therein, wherein the first solution comprises a first polymer species; a second receptacle configured to reversibly retain a second solution therein, wherein the second solution comprises a second polymer species; and a case comprising at least one opening to allow a medical device through, wherein the case is configured to retain the contents of the first and second receptacles for at least 30 minutes.

In any of the preceding embodiments, the first and second polymers species are configured to react in the case to form an at least partially covalently crosslinked hydrogel coating on the medical device.

In any of the preceding embodiments, the kit further comprises the medical device, wherein the medical device comprises a hydrogel layer on a surface of the medical device.

In any of the preceding embodiments, the hydrogel layer is covalently bound to the surface of the medical device and comprises a first hydrophilic polymer and a second hydrophilic polymer that are at least partially covalently cross-linked.

In any of the preceding embodiments, the first hydrophilic polymer is PEG and the second hydrophilic polymer is polyacrylamide.

In any of the preceding embodiments, the first hydrophilic polymer is polyacrylamide and the second hydrophilic polymer is polyacrylamide.

In any of the preceding embodiments, the first hydrophilic polymer comprises a reactive electrophilic group selected from the group consisting of amino-reactive groups, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the second hydrophilic polymer comprises a reactive nucleophilic group selected from the group consisting of amines, amino-reactive groups, sulfhydryl, sulfhydryl-reactive groups, carboxyl groups, hydroxyl groups, haloalkyl groups, dienophile groups, aldehyde or ketone groups, alkenes, epoxides, phosphoramidites, and a combination thereof.

In any of the preceding embodiments, the hydrogel layer on the surface of the medical device comprises an initial thickness before use or wear and a second thickness after use or wear, the second thickness less than the initial thickness, and wherein the first and second solutions are reacted in the case to increase the second thickness to yield a final thickness that is substantially equal to, approaches, or exceeds the initial thickness.

In any of the preceding embodiments, the first solution comprises a PEG polymer species and the second solution comprises a polyacrylamide polymer species.

In any of the preceding embodiments, the first solution comprises a polyacrylamide polymer species and the second solution comprises another polyacrylamide polymer species.

In any of the preceding embodiments, the polyacrylamide polymer species is substantially similar to the other polyacrylamide polymer species.

### Examples

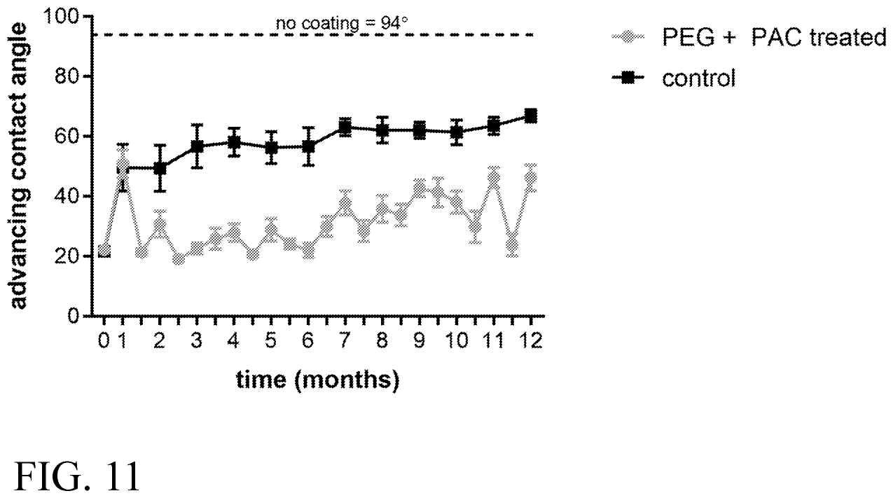

Example 1: Contact Lens Rejuvenation Improves Wetting Angle. Turning now to FIG. 11, monthly treatment with the solutions described herein is shown to improve the advancing contact angle, as measured by a captive bubble test, of treated fluorosilicone acrylate lenses throughout the lifetime of the lens. For comparison, an untreated Optimum lens has an advancing contact angle of 94°, shown as the dotted line in FIG. 11. The control group shows a high advancing contact angle that was maintained substantially around 55-65° while the lenses treated with the rejuvenating solution (PEG and polyacrylamide) had an advancing contact angle of substantially 20° C. that increased over time, during repeated use and reduction of the coating.

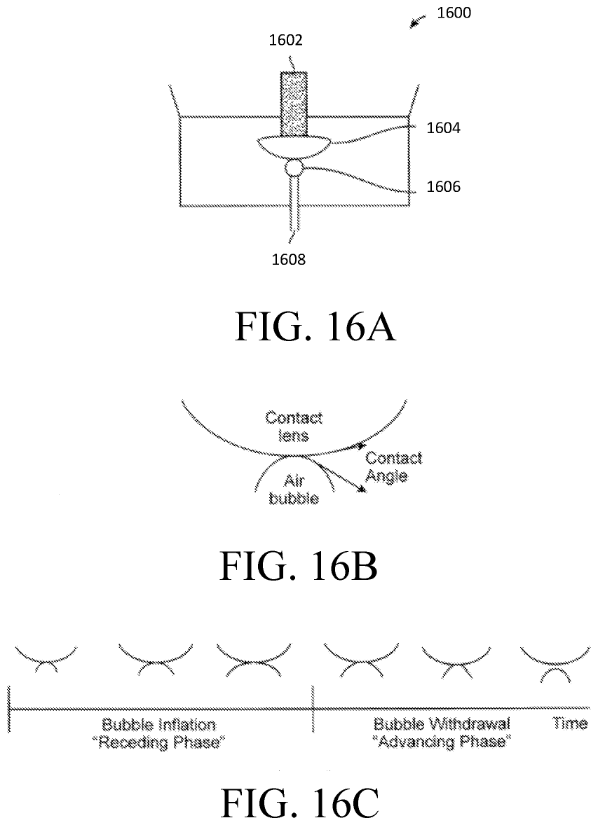

FIGS. 16A-16C show aspects of a captive bubble test that is commonly used in the contact lens industry as a surrogate measure of wettability or hydrophilicity of contact lenses, as provided by embodiments of the technology. FIG. 16A shows the setup 1600 for a captive bubble test. The setup 1600 includes a lens holding fixture 1602 in communication with a test lens 1604. An air bubble 1606 is positioned at a surface of the test lens from a syringe pump 1608.

FIG. 16B shows a schematic view of the contact angle as it occurs in an aqueous solution between the surface of a contact lens and an air bubble, as the air bubble is being inflated against or being withdrawn away from the contact lens.

FIG. 16C provides a schematic series of angles created as a bubble is being inflated against the contact lens surface, and then withdrawn. The left side of the drawing depicts the “receding phase” of the test; the right side of the drawing depicts the “advancing phase” of the test. On the left, after the bubble first makes contact at what will be the central contact point between the bubble and the contact lens, the area of mutual contact expands, and the surrounding aqueous space recedes from the central contact point. Accordingly, this is termed the “receding phase.” On the right, as the bubble is being withdrawn, the aqueous solution advances toward the central point of contact between the bubble and the contact lens. Accordingly, this is termed the “advancing phase” of the test. These profiles can be videographed during the test to capture the dynamics. In the recorded videos, software-based edge detection and angular separation techniques can be used to measure the receding and advancing angles at the interface of the bubble and lens.

In both the advancing and receding portions of the test, a small angle reflects the relatively high affinity of the contact lens surface for water, rather than air. Thus, there is an association between a small contact angle and hydrophilicity or wettability of the contact lens surface. In contrast, a large contact angle reflects a relative lack of affinity of the contact lens surface with water. By means of this test, the hydrophilicity of contact lens embodiments of the technology may be quantified.

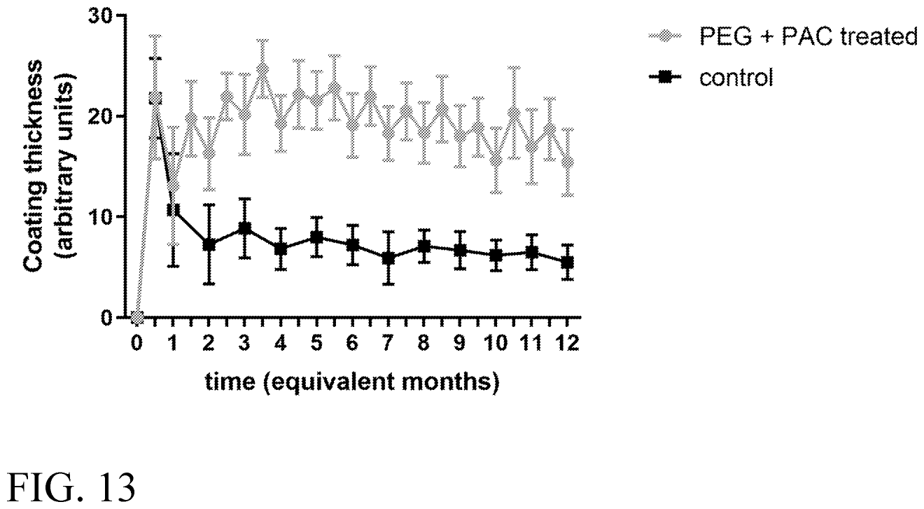

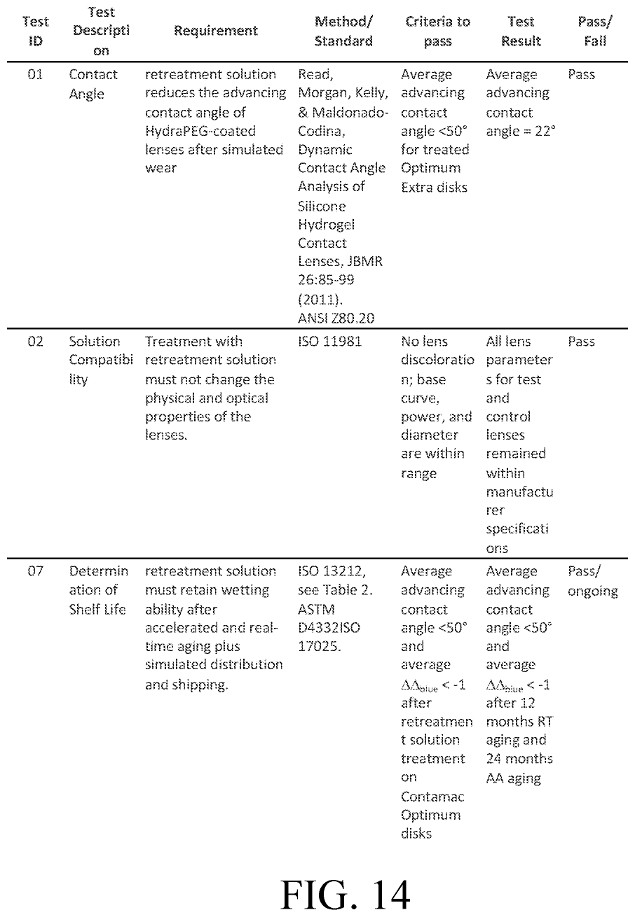

Example 2: Reduction of the Wetting and Contact Angle After Refreshing the Coating on the Contact Lens: A study was conducted to show the viability, efficacy, and safety of the solutions described herein. FIG. 14, for example, shows the results of the study on the reduction of the wetting and contact angle after treatment with the solutions described herein. Contact angles were measured using the captive bubble technique. Receding contact angles were also recorded; however, the more significant changes in contact angle were observed with the advancing angles, indicating high amounts of contact angle hysteresis for lenses on which the PEG and polyacrylamide coating has experienced significant wear.

Turning to FIG. 14, Test 01, the treatment solution was tested for its ability to maintain the wettability of the PEG and polyacrylamide coating. A commercial contact lens was treated with an initial PEG and polyacrylamide coating and then was manipulated (exemplarily by rubbing) to simulate one month of wear. The lens was then treated with the rejuvenating solution (in this example, PEG and polyacrylamide rejuvenating solution). The rejuvenating solution succeeded in maintaining an average advancing contact angle of <50° (i.e., less than 50°), with an average advancing contact angle of 22° overall.

Also in FIG. 14, Test 02, solution compatibility testing was conducted to measure physical parameters (e.g., base curve, power, center thickness, and diameter) of lenses after simulated use or wear. Most lenses, both test and control, were still within the manufacturer's specifications after solution compatibility testing, indicating that the rejuvenating solution is physically compatible with fluorosilicone acrylate lenses.

Also in FIG. 14, Test 07, in order to determine the shelf life of the rejuvenating solution, kits were stored and aging was simulated to test shelf life timepoints of up to 2 years. After treatment of some disks, an advancing contact angle of less than 50° was met for the 12 month, 18 month, and 24 month timepoints.

Example 3: Biocompatibility: Additionally and optionally, the biocompatibility of the treatment solution was tested—component A comprising PEG and polyacrylamide and component B comprising triethanolamine. The PEG and polyacrylamide coating is non-cytotoxic, non-irritating, systemically non-toxic, and showed biocompatible results upon direct ocular exposure for a sub-chronic exposure duration.

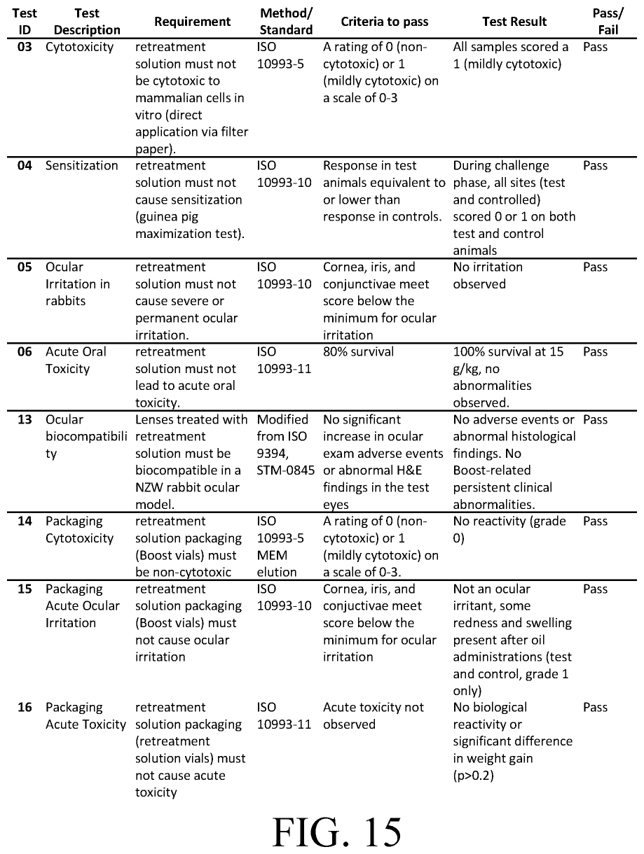

To confirm biocompatibility of the stand-alone PEG and polyacrylamide solution in the form of the rejuvenating solution, cytotoxicity, sensitization, ocular irritation, acute oral toxicity, and ocular biocompatibility testing was performed, as summarized in FIG. 15. This testing was performed directly with the combined treatment solution rather than using extracts from the treated lenses. This represents the worst-case scenario for patient exposure if instructions for use are not followed, as the safety of PEG and polyacrylamide coated lenses has already been demonstrated. Additionally, cytotoxicity, ocular irritation, and acute toxicity of the packaging was tested.

As seen in Test 03 in FIG. 15, rejuvenating solution component A and component B were each separately evaluated for cytotoxicity, with both negative and positive controls. Multiple cell cultures were used as part of the test, and based on qualitative evaluation of the cells thereafter, each of component A and component B were not considered to have a cytotoxic effect.

Additionally, as seen in Test 04 of FIG. 15, sensitization testing on treatment solution component A and the combined components A and B were performed. The skin reactions of the test animals that were exposed to the test article in the induction phase were compared to the reactions of the unexposed negative control animals. No sensitization reactions or patterns were noted in animals exposed to the treatment solution.

Additionally, testing was performed on rejuvenating solution component A to assess ocular irritation. Component A did not elicit an irritation response in this study. Component A was thus considered a non-irritant. Rejuvenating solution component B was not tested because sufficient data has been published indicating that triethanolamine is non-irritating at the concentration used in the solution (0.5 wt %) (“Final Report on the Safety Assessment of Triethanolamine, Diethanolamine, and Monoethanolamine,” 1983).

Additionally, as shown in Test 06 of FIG. 15, oral toxicity to component A was tested. Component A was well tolerated and no toxic signs were observed in any of the tested animals. All major organs appeared healthy at necropsy. Therefore, rejuvenating solution component A, was not considered toxic when administered orally at 15 g/kg to Sprague Dawley rats used in this study. Rejuvenating solution component B was not tested because sufficient data has been published indicating that triethanolamine is non-toxic at the concentration used for this solution (0.5 wt %) (“Final Report on the Safety Assessment of Triethanolamine, Diethanolamine, and Monoethanolamine,” 1983).

Additionally, as seen in Test 05 of FIG. 15, rejuvenating solution was tested in accordance with the product instructions in an ocular biocompatibility study to identify any possible unforeseen issues. As assessed by clinical observations, Draize scoring and McDonald-Shadduck scoring, all eyes were generally normal except for very few, mostly mild, incidental abnormalities (conjunctiva redness, conjunctiva congestion, and/or cornea opacity) prior to and after lens wearing and in eyes fitted with Control Article-treated lenses and with Test Article-treated lenses. On Day 4, all eyes fitted with the Test Article-treated lenses were found normal as assessed by Draize scoring and McDonald-Shadduck scoring. Thus, there were no Test Article-related persistent abnormalities in any of the eyes.

Additionally, as shown in Test 14 of FIG. 15, the cytotoxic potential of extracts of the rejuvenating solution packaging was evaluated, where the test article (blue and orange treatment solution vials) was prepared. Based on qualitative evaluation of the cells exposed to the test article extract, the rejuvenating solution packaging was not considered to have a cytotoxic effect.

Additionally, as shown in Test 15 in FIG. 15, ocular irritation testing of the packaging was conducted. All animals appeared healthy during the course of the study. The packaging was considered a non-irritant to the rabbit eyes.

Additionally, as shown in Test 16 of FIG. 15, acute systemic toxicity testing on the packaging was performed to evaluate systemic responses to test article extracts. Biological reactivity was not observed. No biologically significant differences were noted between the test and control animals. No acute systemic toxicity was observed.

Further, the safety and efficacy of the rejuvenating solution was demonstrated in a clinical trial. Primary safety variables were adverse event reports and discontinuations and detailed slit lamp assessments. The primary efficacy variable was no reduction in lens performance based on visual acuity and lens fit. Both the primary safety and efficacy variables were met in this study.

The usability of the rejuvenating solution was also assessed, and all patients demonstrated an understanding of the treatment's frequency, duration, and other conditions.

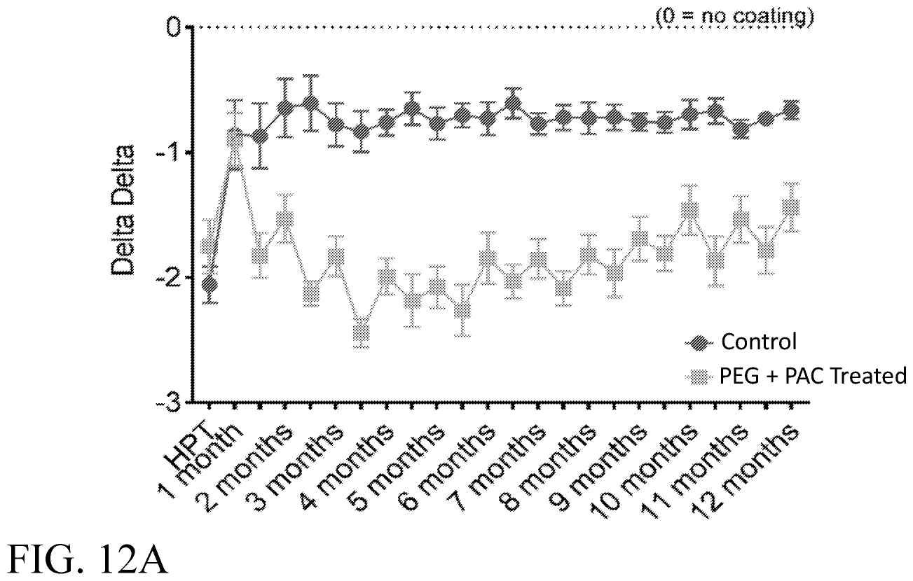

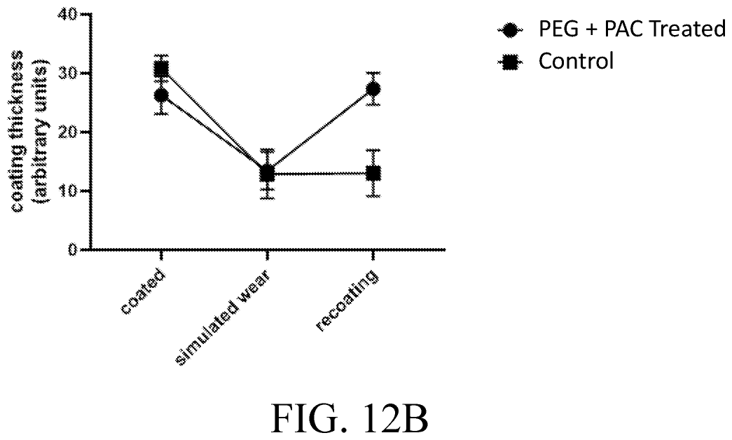

Example 4: Contact Lens Rejuvenation Increases Coating Thickness. Monthly treatment with the solutions described herein is shown to improve the coating thickness, as measured by thin film ellipsometry, of treated fluorosilicone acrylate lenses throughout the lifetime of the lens. For comparison, an untreated Optimum lens has a 0 coating thickness, shown as the dotted line in FIG. 12A. As shown in FIG. 12A, the graph shows delta delta values measured by ellipsometry (FilmSense) where the delta delta is proportional to coating thickness and more negative values indicate a thicker coating. The control group has a reduced coating thickness over time while after treatment with the solutions (PEG and polyacrylamide (PAC) treated) described herein, the coating thickness is maintained using monthly treatments throughout the lens lifetime.

In general, ellipsometry is an indirect method that can be used to determine thickness of a material and other material properties (e.g., composition, roughness, crystalline nature, doping concentration, electrical conductivity, etc.). The measured signal is the change in polarization as the incident radiation (in a known state) interacts with the material structure of interest (reflected, absorbed, scattered, or transmitted). The polarization change is quantified by the amplitude ratio and the phase difference. The signal depends on the thickness as well as the material properties. Additional information about ellipsometry can be found at least in Tompkins, H. and Irene, E. A. “Handbook of Ellipsometry.” 2005, United States of America; William Andrew, Inc., the contents of which are herein incorporated by reference in their entirety. To characterize the thickness of a thin film, the substrate can be measured with and without the film to find delta delta=delta₂−delta₁. The thickness, d, is then directly proportional to the change in delta:

d=C*delta delta

The derivation of this relationship is described by A. N. Saxena “Changes in the phase and amplitude of polarized light reflected from a film-covered surface and their relations with the film thickness,” Journal of the Optical Society of America, 1965; 55(9): 1061-1067, the contents of which are herein incorporated by reference in their entirety.

Example 5: Thickness Testing. FIG. 12B also shows ellipsometry data (similarly measured as in Example 4) as a readout for hydrogel coating thickness for gas permeable contact lens material coated with at least partially cross-linked PEG and polyacrylamide (“coated,” having an initial thickness) and then manually rubbed to wear away some of the coating (“simulated wear,” to achieve a second thickness, less than the initial or baseline thickness). Half of the samples were then recoated with the regenerating solution (“recoating,” PEG and polyacrylamide hydrophilic polymer solution treated (PEG+PAC Treated)), while the other half received no treatment and served as a negative control (Control). The graph shows delta delta values measured by ellipsometry (FilmSense) multiplied by an arbitrary negative value. Delta delta is proportional to coating thickness. Samples recoated with Boost (black circles; PEG and polyacrylamide hydrophilic polymer solution treated) were restored to approximately the same coating thickness (i.e., final thickness) as was initially applied, while no change was observed in the coating thickness of the control samples (black squares; Control).

Example 6: Thickness Over Time. FIG. 13 shows ellipsometry data over time (measured similarly to Examples 4-5) as a readout for hydrogel coating thickness for gas permeable contact lens material coated with at least partially cross-linked PEG and polyacrylamide (having an initial thickness) and then manually rubbed to wear away some of the coating (to achieve a second thickness, less than the initial or baseline thickness). Half of the samples were then recoated with the regenerating solution (PEG and polyacrylamide polymer populations; PEG+PAC treated) over time, while the other half received no treatment and served as a negative control (no regenerating solution applied; Control). The graph shows delta delta values measured by ellipsometry (FilmSense) multiplied by an arbitrary negative value. Delta delta is proportional to coating thickness. Samples recoated with PEG and polyacrylamide polymers (black circles; PEG+PAC treated group) were restored to approximately the initial coating thickness (i.e., final thickness) and maintained this thickness over time with repeated use and subsequent repeated treatment. There was no change observed in the coating thickness of the control samples (black squares; Control). The control group exhibited a significant decrease in thickness with continued use (manual rubbing) and the thickness remained low over time.

Example 7. A medical device comprising an existing coating comprising a first hydrophilic polymer comprising polyacrylamide and a second hydrophilic polymer comprising polyacrylamide treated with a rejuvenating solution comprising a first hydrophilic polymer comprising PEG and a second hydrophilic polymer comprising polyacrylamide. The existing coating not being activated prior to treatment with the rejuvenating solution.

Example 8. A medical device comprising an existing coating comprising a first hydrophilic polymer comprising polyacrylamide and a second hydrophilic polymer comprising polyacrylamide treated with a rejuvenating solution comprising a first hydrophilic polymer comprising polyacrylamide and a second hydrophilic polymer comprising polyacrylamide. The existing coating not being activated prior to treatment with the rejuvenating solution.

Example 9. A medical device comprising an existing coating comprising a first hydrophilic polymer comprising PEG and a second hydrophilic polymer comprising PEG treated with a rejuvenating solution comprising a first hydrophilic polymer comprising PEG and a second hydrophilic polymer comprising polyacrylamide. The existing coating not being activated prior to treatment with the rejuvenating solution.

Example 10. A medical device comprising an existing coating comprising a first hydrophilic polymer comprising PEG and a second hydrophilic polymer comprising PEG treated with a rejuvenating solution comprising a first hydrophilic polymer comprising polyacrylamide and a second hydrophilic polymer comprising polyacrylamide. The existing coating not being activated prior to treatment with the rejuvenating solution.

Example 11. A medical device comprising an existing coating comprising a first hydrophilic polymer comprising PEG and a second hydrophilic polymer comprising polyacrylamide treated with a rejuvenating solution comprising a first hydrophilic polymer comprising polyacrylamide and a second hydrophilic polymer comprising polyacrylamide. The existing coating not being activated prior to treatment with the rejuvenating solution.

Example 12. A medical device comprising an existing coating comprising a first hydrophilic polymer comprising PEG and a second hydrophilic polymer comprising polyacrylamide. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising PEG and a second hydrophilic polymer comprising polyacrylamide.

Example 13. A medical device comprising an existing coating comprising a first hydrophilic polymer comprising polyacrylamide and a second hydrophilic polymer comprising polyacrylamide. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising PEG and a second hydrophilic polymer comprising polyacrylamide.

Example 14. A medical device comprising an existing coating comprising a first hydrophilic polymer comprising polyacrylamide and a second hydrophilic polymer comprising polyacrylamide. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising polyacrylamide and a second hydrophilic polymer comprising polyacrylamide.

Example 15. A medical device comprising an existing coating comprising a first hydrophilic polymer comprising PEG and a second hydrophilic polymer comprising PEG. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising PEG and a second hydrophilic polymer comprising polyacrylamide.

Example 16. A medical device comprising an existing coating comprising a first hydrophilic polymer comprising PEG and a second hydrophilic polymer comprising PEG. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising polyacrylamide and a second hydrophilic polymer comprising polyacrylamide.

Example 17. A medical device comprising an existing coating comprising a first hydrophilic polymer comprising PEG and a second hydrophilic polymer comprising polyacrylamide. The existing coating is activated with pH adjustment (e.g., increasing pH up to 11) in a separate solution or the same solution as the rejuvenating solution. The activated medical device is then treated with a rejuvenating solution comprising a first hydrophilic polymer comprising polyacrylamide and a second hydrophilic polymer comprising polyacrylamide.

Example 18. A medical device comprising an existing coating comprising a first hydrophilic polymer comprising PEG and a second hydrophilic polymer comprising polyacrylamide. The existing coating is activated with light before treatment with the rejuvenating solution. The activated medical device is then treated with a rejuvenating solution comprising a first hydrophilic polymer comprising PEG and a second hydrophilic polymer comprising polyacrylamide.

Example 19. A medical device comprising an existing coating comprising a first hydrophilic polymer comprising polyacrylamide and a second hydrophilic polymer comprising polyacrylamide. The existing coating is activated with light before treatment with the rejuvenating solution. The activated medical device is then treated with a rejuvenating solution comprising a first hydrophilic polymer comprising PEG and a second hydrophilic polymer comprising polyacrylamide.

Example 20. A medical device comprising an existing coating comprising a first hydrophilic polymer comprising polyacrylamide and a second hydrophilic polymer comprising polyacrylamide. The existing coating is activated with light before treatment with the rejuvenating solution. The activated medical device is then treated with a rejuvenating solution comprising a first hydrophilic polymer comprising polyacrylamide and a second hydrophilic polymer comprising polyacrylamide.

Example 21. A medical device comprising an existing coating comprising a first hydrophilic polymer comprising PEG and a second hydrophilic polymer comprising PEG. The existing coating is activated with light before treatment with the rejuvenating solution. The activated medical device is then treated with a rejuvenating solution comprising a first hydrophilic polymer comprising PEG and a second hydrophilic polymer comprising polyacrylamide.

Example 22. A medical device comprising an existing coating comprising a first hydrophilic polymer comprising PEG and a second hydrophilic polymer comprising PEG. The existing coating is activated with light before treatment with the rejuvenating solution. The activated medical device is then treated with a rejuvenating solution comprising a first hydrophilic polymer comprising polyacrylamide and a second hydrophilic polymer comprising polyacrylamide.

Example 23. A medical device comprising an existing coating comprising a first hydrophilic polymer comprising PEG and a second hydrophilic polymer comprising polyacrylamide. The existing coating is activated with light before treatment with the rejuvenating solution. The activated medical device is then treated with a rejuvenating solution comprising a first hydrophilic polymer comprising polyacrylamide and a second hydrophilic polymer comprising polyacrylamide.

Example 24. A medical device comprising an existing coating comprising a first hydrophilic polymer comprising PEG and a second hydrophilic polymer comprising polyacrylamide. The existing coating is wet activated before treatment with the rejuvenating solution. A wet activation process may begin with a lens in a hydrated state. The next step may include activating the hydrated surface lens core by a plasma or chemical treatment. For example, ozone may be used to activate the core surface. Once activated, the activated lens may be placed into a solution containing the coating material. The solution may include a hydrophilic polymer solution as described and water. In some cases, the solution is at a pH>7. The solution may be agitated to create a well-stirred bath and the lens incubates therein. In some cases, the lens incubates for about 50 minutes. Next, the lens may be transferred to a water bath to equilibrate in water. The equilibration step may also serve to wash excess polymer from the lens. The lens may be equilibrated in water for about 50 minutes. The lens may be transferred to a packaging container with packaging solution. Additionally, as another step, the lens may be autoclaved. In some cases, the lens is autoclaved at about 250° F. for about 30 minutes. After the autoclave step, the resulting coated lens is ready for use. The method may be understood as an immersive method, wherein activated lens cores are immersed in a reaction solution within a stirred vessel, the solution including hydrophilic macromer reactants, and the reaction vessel operated to achieve appropriate reaction conditions. The reaction vessel and aspects of the conditions, in biochemical engineering terms, may be understood as occurring in a continuously stirred reaction tank (CSTR). In typical embodiments, the reacting steps occur within a reaction solution that has an aqueous solvent. Such the aqueous solvent may include any one or more of water, methanol, ethanol, or any suitable aqueous solvent that solubilizes PEG. The wet activated medical device is then treated with a rejuvenating solution comprising a first hydrophilic polymer comprising PEG and a second hydrophilic polymer comprising polyacrylamide.

Example 25. A medical device comprising an existing coating comprising a first hydrophilic polymer comprising polyacrylamide and a second hydrophilic polymer comprising polyacrylamide. The existing coating is wet activated, as in Example 24, before treatment with the rejuvenating solution. The activated medical device is then treated with a rejuvenating solution comprising a first hydrophilic polymer comprising PEG and a second hydrophilic polymer comprising polyacrylamide.

Example 26. A medical device comprising an existing coating comprising a first hydrophilic polymer comprising polyacrylamide and a second hydrophilic polymer comprising polyacrylamide. The existing coating is wet activated, as in Example 24, before treatment with the rejuvenating solution. The activated medical device is then treated with a rejuvenating solution comprising a first hydrophilic polymer comprising polyacrylamide and a second hydrophilic polymer comprising polyacrylamide.

Example 27. A medical device comprising an existing coating comprising a first hydrophilic polymer comprising PEG and a second hydrophilic polymer comprising PEG. The existing coating is wet activated, as in Example 24, before treatment with the rejuvenating solution. The activated medical device is then treated with a rejuvenating solution comprising a first hydrophilic polymer comprising PEG and a second hydrophilic polymer comprising polyacrylamide.

Example 28. A medical device comprising an existing coating comprising a first hydrophilic polymer comprising PEG and a second hydrophilic polymer comprising PEG. The existing coating is wet activated, as in Example 24, before treatment with the rejuvenating solution. The activated medical device is then treated with a rejuvenating solution comprising a first hydrophilic polymer comprising polyacrylamide and a second hydrophilic polymer comprising polyacrylamide.

Example 29. A medical device comprising an existing coating comprising a first hydrophilic polymer comprising PEG and a second hydrophilic polymer comprising polyacrylamide. The existing coating is wet activated, as in Example 24, before treatment with the rejuvenating solution. The activated medical device is then treated with a rejuvenating solution comprising a first hydrophilic polymer comprising polyacrylamide and a second hydrophilic polymer comprising polyacrylamide.

Example 30. A medical device comprising an existing coating comprising a first hydrophilic polymer comprising PEG and a second hydrophilic polymer comprising polyacrylamide. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising PEG and a second hydrophilic polymer comprising polyacrylamide.

Example 31. A medical device comprising an existing coating comprising a first hydrophilic polymer comprising polyacrylamide and a second hydrophilic polymer comprising polyacrylamide. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising PEG and a second hydrophilic polymer comprising polyacrylamide.

Example 32. A medical device comprising an existing coating comprising a first hydrophilic polymer comprising polyacrylamide and a second hydrophilic polymer comprising polyacrylamide. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising polyacrylamide and a second hydrophilic polymer comprising polyacrylamide.

Example 33. A medical device comprising an existing coating comprising a first hydrophilic polymer comprising PEG and a second hydrophilic polymer comprising PEG. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising PEG and a second hydrophilic polymer comprising polyacrylamide.

Example 34. A medical device comprising an existing coating comprising a first hydrophilic polymer comprising PEG and a second hydrophilic polymer comprising PEG. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising polyacrylamide and a second hydrophilic polymer comprising polyacrylamide.

Example 35. A medical device comprising an existing coating comprising a first hydrophilic polymer comprising PEG and a second hydrophilic polymer comprising polyacrylamide. The existing coating is activated by adding a monomer that reacts with the existing coating on the medical device that leaves reactive sites for one or more reactive groups in the rejuvenating solution. The activated medical device is then treated with a rejuvenating solution comprising a first hydrophilic polymer comprising polyacrylamide and a second hydrophilic polymer comprising polyacrylamide.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a substance, or composition.

As used herein, the term “comprising” or “comprises” is intended to mean that the compositions, kits, systems, and methods include the recited elements, and may additionally include any other elements. “Consisting essentially of” shall mean that the compositions, kits, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the compositions, kits, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-29. (canceled)

30. A method for regenerating a coating on a surface of a hydrogel coated contact lens, wherein an initial thickness of an original hydrogel coating is greater than a second thickness of the original hydrogel coating after use or wear, the method comprising:
providing a first solution comprising a polyethylene glycol (PEG) polymer species comprising 2 to 12 branch arms, wherein one or more of the branch arms comprise a terminal sulfonyl reactive group;
providing a second solution comprising a polyacrylamide polymer species comprising one or more pendant amine groups along a backbone of the polyacrylamide polymer species;
reacting the first and second solutions such that the terminal sulfonyl reactive group of the PEG and the one or more pendant amine groups of the polyacrylamide react with each other and with one or more of an available: amine, sulfonyl, carboxyl, or hydroxyl on the surface of the hydrogel coated contact lens having the second thickness, wherein the terminal sulfonyl reactive group is in a molar excess of 0.5 to 4 times of the one or more pendant amine groups; and
increasing the second thickness of the original hydrogel coating on the contact lens so that a final thickness of the regenerated coating approaches the initial thickness,
wherein the final thickness and the initial thickness are each about 25 nm to about 100 nm.

31. The method of claim 30, wherein reacting further comprises forming an at least partially covalently crosslinked hydrogel coating on the contact lens.

32. The method of claim 30, wherein the original hydrogel coating is on a surface of the contact lens.

33. The method of claim 30, further comprising providing the contact lens with the original hydrogel coating covalently bound to the surface, wherein the original hydrogel coating comprises a first polymer species and a second polymer species that are at least partially covalently cross-linked.

34. The method of claim 30, wherein the final thickness substantially equals the initial thickness.

35. The method of claim 30, further comprising:
removing protein and lipid deposits from the contact lens having the hydrogel coating having the second thickness after use or wear, wherein removing protein and lipid deposits from the contact lens does not activate an outer surface of the contact lens.

36. (canceled)

37. The method of claim 30, further comprising incubating the contact lens in the first and second solutions at room temperature for a predetermined length of time.

38. The method of claim 37, wherein the predetermined length of time is 15 to 45 minutes.

39. The method of claim 37, wherein the incubating step occurs between 20 to 40° C.

40. The method of claim 37, wherein the incubating step occurs between 20 to 25° C.

41. (canceled)

42. The method of claim 30, wherein the second thickness is between 5 nm and 80 nm.

43-49. (canceled)

50. The method of claim 30, further comprising forming the original hydrogel layer on the contact lens, wherein forming comprises:
reacting an outer surface of the contact lens with a first polymer species of a hydrophilic polymer solution, wherein the first polymer species comprises a moiety at a first portion that forms a covalent attachment to the outer surface of the contact lens; and
reacting the first polymer species of the hydrophilic polymer solution with a second polymer species of the hydrophilic polymer solution, the second polymer species comprising a moiety that forms a covalent bond to a second portion of the first polymer species in a second covalent reaction thereby forming the original hydrogel coating comprising the first polymer species and the second polymer species at least partially cross-linked.

51. The method of claim 50, wherein the moiety at the first portion, the second portion, or both of the first polymer species comprises a reactive sulfonyl group.

52. The method of claim 51, wherein the moiety of the second polymer species comprises a reactive amino group.

53. The method of claim 50, wherein the first polymer species comprises PEG or polyacrylamide.

54. The method of claim 53, wherein the second polymer species comprises PEG or polyacrylamide.

55. The method of claim 50, wherein the moiety at the first portion, the second portion, or both of the first polymer species comprises a reactive carboxyl group.

56. The method of claim 55, wherein the moiety of the second polymer species comprises a reactive amino group.

57-93. (canceled)

94. A method for regenerating a coating on a surface of a hydrogel coated contact lens, wherein an initial thickness of an original hydrogel coating is greater than a second thickness of the original hydrogel coating after use or wear, the method comprising:
providing a first solution comprising a polyethylene glycol (PEG) polymer species comprising one or more of the branch arms comprising a sulfonyl reactive group;
providing a second solution comprising a polyacrylamide polymer species comprising one or more amine groups;
reacting the first and second solutions such that the sulfonyl reactive group of the PEG and the one or more amine groups of the polyacrylamide react with each other and with one or more of an available: amine, sulfonyl, carboxyl, or hydroxyl on the surface of the hydrogel coated contact lens having the second thickness, wherein the sulfonyl reactive group is in a molar excess of 0.5 to 4 times of the one or more amine groups; and
increasing the second thickness of the original hydrogel coating on the contact lens so that a final thickness of the regenerated coating approaches the initial thickness,
wherein the final thickness and the initial thickness are each about 25 nm to about 100 nm.

95. The method of claim 94, further comprising incubating the contact lens in the first and second solutions at a temperature between 20 to 40° C.
